(19) United States
(12) Patent Application Publication
(10) Pub. No.: US 2023/0346380 A1
(43) Pub. Date: Nov. 2, 2023

(54) METHODS AND DEVICES FOR THE TREATMENT OF PULMONARY DISORDERS WITH A BRAIDED IMPLANTABLE FLOW CONTROL DEVICE

(71) Applicant: EOLO MEDICAL INC., New York, NY (US)

(72) Inventors: Sean TOTTEN, Kirkland, WA (US); Residenc LEE, Milpitas, CA (US); Joe M. GUERRICABEITIA, Shoreline, WA (US); Don TANAKA, Saratoga, CA (US); Abha Prashant CHINUBHA, Fremont, CA (US); Mark S. LEUNG, New York, NY (US); Lucas SANTANNA, New York, NY (US); Claudia PHAM, NewYork, NY (US); Abigail BRAZIL, New York, NY (US)

(21) Appl. No.: 17/794,944

(22) PCT Filed: Jan. 22, 2021
(86) PCT No.: PCT/US2021/014581

§ 371 Date: Jul. 22, 2022

Related U.S. Application Data
(60) Provisional application No. 62/964,370, filed on Jan. 22, 2020.

(51) Int. Cl. A61B 17/12; A61B 90/00

(52) U.S. Cl. CPC A61B 17/12104; A61B 17/12036; A61B 17/12145; A61B 90/08; A61B 2017/12095; A61B 2090/0811

(57) ABSTRACT

A flow control device (300, 324, 330) for a bronchial passageway including: a flow control valve (307, 335); a braided wire structural frame (303) expandable from a collapsed configuration to an expanded configuration, in the collapsed configuration the frame is an extended tube and in the collapsed configuration the frame includes a wall contact section (310), a middle support section (312) within the wall contact section, and a fold (311) between and connecting the wall contact section and the middle support section; and a sealing membrane (305) mounted to at least a distal portion of the structural frame, wherein the sealing membrane forms an enclosed wall defining at least a portion of an airflow passage through the flow control device, and the flow control valve is included in the airflow passage and extending inward from the enclosed wall and at least partially within the wall contact section.

### RELATED APPLICATION

This application claims priority to U.S. Provisional application 62/964,370 filed Jan. 22, 2020, the entirety of which is incorporated by reference.

### TECHNICAL FIELD

The field of the invention is lung volume reduction devices used to treat hyper-inflated lung, for example in patients diagnosed with chronic obstructive pulmonary disease (COPD), emphysema, asthma, bronchitis. The invention relates to lung volume reduction devices such as deployable valves configured to be delivered through the airway to the lung with minimally invasive techniques.

### BACKGROUND

Hyper-inflated lung is a lung disease that makes it hard to breathe. COPD is a major cause of disability and is the third leading cause of death in the United States. The symptoms and effects of COPD often worsen over time, such as over years, and can limit the ability of a person suffering from COPD to do routine activities. Current medical techniques offer no solution for reversing the damage to the airways and lungs associated with COPD.

COPD often does not affect all air sacs or alveoli equally in a lung. A lung may have diseased regions in which the air sacs are damaged and unsuited for gas exchange. The same lung may have healthy regions (or at least relatively healthy regions) in which the air sacs continue to perform effective gas exchange. The diseased regions may be large, such as 20 to 30 percent or more of the lung volume.

The diseased regions of the lung occupy volume in the pulmonary cavity, which could otherwise be occupied by the healthy portion of the lung. If the healthy regions(s) of the lung were allowed to expand into the volume occupied by the diseased regions, the healthy regions could expand and fill with air to allow the air sacs in the healthy region to exchange oxygen for carbon dioxide.

In U.S. Patent Application Publication 2014/0058433 describes methods and devices are adapted for regulating fluid flow to and from a region of a patient's lung, such as to achieve a desired fluid flow dynamic to a lung region during respiration and/or to induce collapse in one or more lung regions. Pursuant to an exemplary procedure, an identified region of the lung is targeted for treatment. The targeted lung region is then bronchially isolated to regulate airflow into and/or out of the targeted lung region through one or more bronchial passageways that feed air to the targeted lung region.

U.S. Pat. No. 7,842,061 discloses an intra-bronchial device placed and anchored in an air passageway of a patient to collapse a lung portion associated with the air passageway. The device includes a support structure, an obstructing member carried by the support structure that reduces ventilation to the lung portion by preventing air from being inhaled into the lung portion, and at least one anchor carried by the support structure that anchors the obstruction device within the air passageway. The anchor may engage the air passageway wall by piercing or friction, include a stop dimensioned for limiting the piercing of the air passageway wall, and may be releasable from the air passageway for removal of the intra-bronchial device. The anchors may be carried by a peripheral portion of the support structure, or by a central portion of the support structure. The obstructing member may be a flow control valve.

International Publication Number WO 2004/010845 discloses a flow control device for a bronchial passageway. The device can include a valve member that regulates fluid flow through the flow control device, a frame coupled to the valve member, and a membrane attached to the frame. At least a portion of the flow control device forms a seal with the interior wall of the bronchial passageway when the flow control device is implanted in the bronchial passageway. The membrane forms a fluid pathway from the seal into the valve member to direct fluid flowing through the bronchial passageway into the valve member.

However, there remains a need for a lung volume reduction device and procedure that effectively treats patients suffering from a hyperinflated lung that is also affordable, quick to implant, easily assessable and removable, and safe.

### SUMMARY

This disclosure is related to methods, devices, and systems for reducing volume of a hyper-inflated lung, for example in a patient suffering from COPD.

One aspect of the disclosure is a device for reducing volume of a patient's diseased lung lobe comprising a proximal end, a distal end, a deployable structural frame, a sealing element, a valve, and a retention element. The device may be embodied as an endobronchial valve, such as a lobar flow control valve. These functions may be served by distinct structures or in some embodiments one or more structures may provide one or more of these functions.

The structural frame may further comprise a coupler on its proximal end. The coupler may be configured to mate with a delivery tool and transmit torque and translation applied to the delivery tool to the device.

The endobronchial valve, such as a lobar flow control valve, may have a sealing element that is a flexible membrane connected to the structural frame.

The endobronchial valve may include a flow control valve that permits air to flow in a direction from the distal end to the proximal end.

Also disclosed herein is a method of treating a patient with COPD comprising delivering a lobar valve through a working channel of a bronchoscope and deploying the lobar valve in a lobar bronchus that feeds a diseased lobe of the patient's lungs so that the lobar valve permits air to be released from the diseased lobe and air is not permitted to pass into the diseased lobe. The method may further comprise affixing a retention element of the lobar valve to an airway carina distal to the lobar bronchus. The retention element may be an airway carina screw or an airway carina clip. The valve may be positioned in the lobar bronchus such that the axis of the valve is not parallel with the axis of the lobar bronchus.

One or more further aspects of the disclosure are provided below.

A first aspect relates to a flow control device for a bronchial passageway comprising: a flow control valve; a braided wire structural frame, wherein the structural frame is expandable from a collapsed configuration to an expanded configuration, and in the collapsed configuration the frame is an extended tube and in the collapsed configuration the frame includes a wall contact section, a middle support section within the wall contact section, and a fold between and connecting the wall contact section and the middle support section; and a sealing membrane mounted to at least a distal portion of the structural frame, wherein the sealing membrane forms an enclosed wall defining at least a portion of an airflow passage through the flow control device, and the flow control valve is included in the airflow passage and extending inward from the enclosed wall and at least partially within the wall contact section.

A 2^{nd} aspect relates to the flow control device of the first aspect, wherein the flow control valve is integrated in the sealing membrane.

A 3^{rd} aspect relates to the flow control device of the 1^{st} or 2^{nd} aspect, wherein the flow control device further comprises a coupler and spokes extending radially outward from the coupler to a proximal end of the braided wire structural frame.

A 4^{th} aspect relates to the flow control device of the 1^{st} to 3^{rd} aspects, wherein the wall contact section is longer than the middle support section.

A 5^{th} aspect relates to the flow control device of the 1^{st} to 3^{rd} aspects, wherein the braded wire structural frame includes an inner support section connected to the middle support section by a second fold.

A 6^{th} aspect relates to the flow control device of the 5^{th} aspect, wherein the middle support section is longer than the inner support section.

A 7^{th} aspect relates to the flow control device of the 5^{th} to 6^{th} aspects, wherein the inner support section is directly connected to spokes extending radially inward of the inner support section to a coupler.

An 8^{th} aspect relates to the flow control device of the 1^{st} to 7^{th} aspects, wherein a width of the braided wire structural frame, in the expanded configuration is in a range of 7 mm to 12 mm or in a range of 5 mm to 15 mm or in a range of 11 mm to 14 mm or in a range of 10 mm to 18 mm.

A 9^{th} aspect relates to the flow control device of the 1^{st} to 8^{th} aspects, wherein a length of the flow control device in the expanded configuration is in a range of 8 mm to 18 mm.

A 10^{th} aspect relates to the flow control device of the 1^{st} to 9^{th} aspects, wherein the structural frame, while in the expanded configuration, includes a cylindrical airway wall contact section.

An 11^{th} aspect relates to the flow control device of the 10^{th} aspect, wherein at least a part of the sealing membrane is bonded to the cylindrical airway contact section.

A 12^{th} aspect relates to the flow control device of the 10^{th} aspect, wherein the sealing member covers the cylindrical airway wall contact section and spokes included in the structural frame.

A 13^{th} aspect relates to the flow control device of the 1^{st} to 12^{th} aspects, wherein the structural frame, in the collapsed configuration has a diameter no greater than 2.6 mm.

A 14^{th} aspect relates to the flow control device of the 1^{st} to 13^{th} aspects, wherein the structural frame, in the collapsed configuration has a diameter in a range of 2 mm to 2.6 mm.

A 15^{th} aspect relates to the flow control device of the 1^{st} to 14^{th} aspects, wherein a ratio of a length to a width of the structural frame in the expanded configuration is in a range of 0.28:1 to 0.54:1, such as about 0.417:1.

A 16^{th} aspect relates to the flow control device of the 1^{st} to 14^{th} aspects, wherein a ratio of a width of the structural frame in the expanded configuration to the width in the collapsed configuration is in a range of 4:1 to 7:1, such as about 5.45:1.

A 17^{th} aspect relates to the flow control device of the 1^{st} to 16^{th} aspects, wherein the flow control device includes a coupler at a proximal end of the device.

An 18^{th} aspect relates to the flow control device of the 1^{st} to 17^{th} aspects, wherein the flow control device includes a coupler at a proximal end of the device, and the coupler is configured to connected to a corresponding coupler of a shaft of a delivery device.

An 19^{th} aspect relates to the flow control device of the 18^{th} aspect, wherein the coupler is formed from a laser cut tube.

A 20^{th} aspect relates to the flow control device of the 19^{th} aspect, wherein the laser cut tube has a wall thickness in a range of 0.11 mm to 0.17 mm.

A 21^{st} aspect relates to the flow control device of the 19^{th} aspect, wherein the laser cut tube also forms spokes connected to the braided wire structural frame.

A 22^{nd} aspect relates to the flow control device of the 1^{st} to 21^{st} aspects, wherein the sealing membrane has a micropattern molded at least on the exterior surface of the airway wall contact section, the micropattern configured to be hydrophilic.

A 23^{rd} aspect relates to the flow control device of the 1^{st} to 22^{nd} aspects, wherein sealing membrane has a micropattern molded on at least one of the interior surface of the airway contact section and the flow control valve, the micropattern configured to increase hydrophobic nature of the sealing membrane.

A 24^{th} aspect relates to the flow control device of the 1^{st} to 23^{rd} aspects, wherein the ratio of the diameter of the flow control device in the collapsed configuration to the diameter of the flow control device in the expanded configuration is in a range of 1:10 to 2:10

A 25^{th} aspect relates to the flow control device of the 1^{st} to 24^{th} aspects, wherein the delivery length is in a range of 30 to 40 mm and the deployed length is in a range of 8 to 18 mm.

A 26^{th} aspect relates to the flow control device of the 1^{st} to 25^{th} aspects, further comprising at least one anti-inversion feature within the flow control valve.

A 27^{th} aspect relates to the flow control device of the 26^{th} aspect, wherein the anti-inversion feature is a at least one joint, such as a weld, between opposite lips of the flow control valve.

A 28^{th} aspect relates to the flow control device of the 26^{th} or 27^{th} aspects, wherein the at least one anti-inversion feature occupies only portion of a width between opposite side edges of the lips of the flow control device.

A 29^{th} aspect relates to the flow control device of the 26^{th} to 28^{th} aspects, wherein the at least one anti-inversion feature cumulatively occupies a width in a range of 5% to 25% of a width between opposite side edges of the lips of the flow control device.

A 30^{th} aspect relates to the flow control device of the 26^{th} to 29^{th} aspects, wherein each of the at least one anti-inversion feature has a width in a range of 0.3 to 1.5 mm and the flow control valve has a width in a range of 7 mm to 10 mm.

A 31^{st} aspect relates to the flow control device of the 26^{th} to 30^{th} aspects, wherein the at least one anti-inversion feature joins opposite lips of the flow control valve at the distal ends of the lips.

A 32^{nd} aspect relates to the flow control device of the 26^{th} to 31^{st} aspects, wherein the at least one anti-inversion feature is in a range of 0.5 to 3 mm from a plane of an inlet to the flow control valve.

A 33^{rd} aspect relates to the flow control device of the 26^{th} to 32^{nd} aspects, wherein the flow control device includes opposing lips, and an inside surface of at least one of the lips is dimpled in a region proximate to the at least one anti-inversion feature.

A 34^{th} aspect relates to the flow control device of the 1^{st} to 34^{th} aspects, wherein the wall contact section, in the expanded configuration, is oval in cross-section, and the flow control valve is elongated in cross-section.

A 35^{th} aspect relates to the flow control device of the 34^{th} aspect, wherein major axes of the cross-section of the wall contact section and of the flow control valve are parallel.

A 36^{th} aspect relates to the flow control device of the 34^{th} to 35^{th} aspects, wherein semi-major axes of the cross-section of the wall contact section and of the flow control valve are parallel.

A 37^{th} aspect relates to the flow control device of the 34^{th} to 36^{th} aspects, wherein opposite lips of the flow control valve are aligned with the major axis.

A 38^{th} aspect relates to the flow control device of the 34^{th} to 37^{th} aspects, wherein the flow control device parts along a line parallel to the major axis.

A 39^{th} aspect relates to the flow control device of the 34^{th} to 38^{th} aspects, wherein a surface area of a portion of the sealing membrane spanning a distal end of the braided wire structural frame is in a range of 5% to 15% greater than an area circumscribed by the distal end of the braided wire structural frame in the expanded configuration.

A 40^{th} aspect relates to the flow control device of the 1^{st} to 39^{th} aspects, wherein a ring surrounds the flow control valve and the ring is integral with the sealing membrane.

A 41^{st} aspect relates to the flow control device of the 40^{th} aspect, wherein the ring has a stiffness greater than a stiffness of the sealing membrane radially outward of the ring.

A 42^{nd} aspect relates to the flow control device of the 40^{th} to 41^{st} aspects, wherein the ring has a thickness greater than a thickness of the sealing membrane radially outward of the ring.

A 43^{rd} aspect relates to an assembly of an air flow control device and an insertion tool for a bronchial passageway comprising: air flow control device, wherein each of the air flow control devices includes: a flow control valve; a braided wire structural frame, wherein the structural frame is expandable from a collapsed configuration to an expanded configuration and the braided wire structural frame in the collapsed configuration is an elongated tube and in the expanded configuration includes a wall contact section, a middle support section residing radially within the wall contact section, and a first fold between the wall contact section and the middle support section; a sealing membrane mounted to at least a distal portion of the structural frame, wherein the sealing membrane forms an enclosed wall defining at least a portion of an airflow passage through the flow control device, and the flow control valve is included in the airflow passage, and a first coupler at a proximal end of the airflow control device; a delivery sheath configured to be positioned in a bronchial passageway, wherein the delivery sheath includes a distal end, wherein the air flow control device, while in the collapsed configuration, is within the delivery sheath; a delivery shaft within the delivery sheath and extends through the delivery sheath towards the distal end; and a second coupler at the distal end of the delivery shaft, wherein the second coupler is configured to securely engage the first coupler, wherein the delivery shaft is configured to advance through the delivery sheath to push the air flow control device from the distal end of the delivery sheath and into the bronchial passageway, wherein the air flow control device is configured to expand from the collapsed configuration into the expanded configuration after the air flow control device is pushed out of the delivery sheath, and wherein the air flow control device is configured to automatically release from the second coupler when an actuator on a handle of the assembly is actuated.

A 44^{th} aspect relates to the assembly of aspect 43, further comprising a visual marker on a distal region of the delivery sheath, wherein the visual marker indicates an angular position of a semi-minor or semi-major axis of the braided wire structural frame.

A 45^{th} aspect relates to an implantable airflow control device for a lobar bronchus comprising a distal end and a proximal end, a braided Nitinol frame, and a membrane affixed to a distal end of the frame, and wherein the airflow control device expands from a collapsed state to an expanded state, and the frame in the collapsed configuration is an elongated tube and in the expanded configuration includes a wall contact section, a middle support section residing radially within the wall contact section, and a first fold between the wall contact section and the middle support section.

A 46^{th} aspect relates to the device of aspect 45, wherein the braided frame, comprises a wall contact section, a first fold, a middle support section residing radially within the wall contact section, and a second fold.

A 47^{th} aspect relates to the device of aspect 46, wherein the middle support section resides radially within the wall contact section when the device is in its expanded state and adjacent to the wall-contact section when the device is in its collapsed state.

A 48^{th} aspect relates to the device of the 46^{th} or 47^{th} aspects, wherein the middle support section is shorter than the wall contact section.

A 49^{th} aspect relates to the device of the 46^{th} or 47^{th} aspects, further comprising an inner support section.

A 50^{th} aspect relates to the device of the 49^{th} aspects, wherein the inner support section resides radially within the middle support section when the device is in its expanded state and adjacent to the middle support section when the device is in its collapsed state.

A 51^{st} aspect relates to the device of the 49^{th} or 50^{th} aspects, wherein the inner support section is shorter than the middle support section.

A 52^{nd} aspect relates to the device of the 46^{th} to 51^{st} aspects, wherein the braided Nitinol frame comprises a first braid angle in at least the wall-contact section and a second braid angle in at least the first fold.

A 53^{rd} aspect relates to the device of the 52^{nd} aspect, wherein the first braid angle is less than the second braid angle.

A 54^{th} aspect relates to the device of the 46^{th} to 53^{rd} aspects, wherein the wall-contact section has a length in a range of 8 mm to 18 mm when the device is in its expanded state.

A 55^{th} aspect relates to the device of the 46^{th} to 54^{th} aspects, wherein the device has a diameter in a range of 7 mm to 12 mm or in a range of 5 mm to 15 mm or in a range of 11 mm to 14 mm or in a range of 10 mm to 18 mm when the device is in its expanded state.

A 56^{th} aspect relates to the device of the 46^{th} to 55^{th} aspects, wherein the device has a diameter in a range of 2 to 2.6 mm in its collapsed state.

A 57^{th} aspect relates to the device of the 46^{th} to 56^{th} aspects, wherein the device has a length to diameter ratio in a range of 0.28:1 to 0.54 to 1 in its expanded state.

A 58^{th} aspect relates to the device of the 46^{th} to 57^{th} aspects, wherein the device has a diameter in the expanded state to a diameter in the collapsed state ration in a range of 4:1 to 7:1, such as about 5.45:1.

A 59^{th} aspect relates to the device of the 46^{th} to 58^{th} aspects, wherein the braided Nitinol frame comprises closed loop ends at its distal end.

A 60^{th} aspect relates to the device of the 59^{th} aspects, wherein the closed loop ends are bent inward toward a central axis of the device in its expanded state.

A 61^{st} aspect relates to the device of the 58^{th} to 60^{th} aspects, in combination with aspect 46, wherein the closed loop ends have an angle that is less than a braid angle of the wall-contact section.

A 62^{nd} aspect relates to the device of the 58^{th} to 61^{st} aspects in combination with aspect 46, wherein at least a portion of the closed loop ends alternate in length.

A 63^{rd} aspect relates to the device of the 47^{th} to 62^{nd} aspects, wherein the first fold has a bend radius in a range of 0.75 mm+/−0.5 mm.

A 64^{th} aspect relates to the device of the 46^{th} to 63^{rd} aspects, wherein at least a portion of wires forming the braided Nitinol frame are connected to a coupler.

A 65^{th} aspect relates to the device of the 64^{th} aspect, wherein at least a portion of the wires connected to the coupler are bound together to form spokes.

A 66^{th} aspect relates to the device of the 65^{th} aspect, wherein the device comprises 3 to 15 spokes.

A 67^{th} aspect relates to the device of the 64^{th} to 66^{th} aspects, wherein each pair of adjacent spokes define a space.

A 68^{th} aspect relates to the device of the 67^{th} aspect, wherein each space has an area in a range of 5 mm² to 40 mm².

A 69^{th} aspect relates to the device of the 65^{th} to 68^{th} aspects, wherein the spokes have lengths in a range of 5 to 15 mm.

A 70^{th} aspect relates to the device of the 65^{th} to 69^{th} aspects, wherein the spokes have lengths in a range between a radius of the targeted bronchus to 3 mm more than the maximum diameter of the targeted bronchus.

A 71^{st} aspect relates to the device of the 65^{th} to 70^{th} aspects, wherein all of the spokes have equal lengths.

A 72^{nd} aspect relates to the device of the 65^{th} to 71^{st} aspects, wherein the spokes have a shape set S-curve.

A 73^{rd} aspect relates to the device of the 65^{th} to 72^{nd} aspects, wherein the braided Nitinol frame comprises at least one Nitinol wire.

A 74^{th} aspect relates to the device of the 73^{rd} aspect, wherein the Nitinol wire has a diameter in a range of 0.003″ to 0.007″.

A 75^{th} aspect relates to the device of the 73^{rd} to 74^{th} aspects, wherein the Nitinol wire has a transition temperature less than 32° C.

A 76^{th} aspect relates to the device of the 46^{th} to 75^{th} aspects, further comprising barbs.

A 77^{th} aspect relates to the device of the 46^{th} to 76^{th} aspects, further comprising barbs protruding radially outward from the braided Nitinol frame while in the expanded state and not protruding radially outward from the structural frame while in the collapsed state.

A 78^{th} aspect relates to the device of the 77^{th} aspect, wherein the barbs extend at an angle acute to a longitudinal axis of the device.

A 79^{th} aspect relates to the device of the 77^{th} to 78^{th} aspects, wherein some of the barbs are angled towards a distal end of the flow control device and others of the barbs are angled towards a proximal end of the flow control device.

An 80^{th} aspect relates to the device of the 77^{th} to 79^{th} aspects, in combination with aspect 47, wherein at least some of the barbs extend from spokes of the structural frame.

An 81^{st} aspect relates to the device of the 77^{th} to 80^{th} aspects, in combination with aspect 46, wherein at least some of the barbs extend from the wall-contact section of the frame.

An 82^{nd} aspect relates to the device of the 65^{th} to 81^{st} aspects, wherein the coupler is positioned at the proximal end of the device, and the coupler is configured to connected to a corresponding coupler of a shaft of a delivery device.

An 83^{rd} aspect relates to the device of the 82^{nd} aspect, wherein the coupler comprises a threaded lumen.

An 84^{th} aspect relates to the device of the 46^{th} to 83^{rd} aspects, further comprising a flow control valve.

An 85^{th} aspect relates to the device of the 46^{th} to 84^{th} aspects, wherein the membrane comprises a wall-contact section and a flow control valve.

An 86^{th} aspect relates to the device of the 84^{th} to 85^{th} aspects, wherein the flow control valve is positioned at the distal end of the device.

An 87^{th} aspect relates to the device of the 46^{th} to 86^{th} aspects, wherein the membrane further comprises a lumen occluding section between the wall-contact section and the flow control valve.

An 88^{th} aspect relates to the device of the 46^{th} to 87^{th} aspects, wherein the membrane comprises a hydrophilic micropattern surface at least on the external surface of the wall-contact section.

An 89^{th} aspect relates to the device of the 46^{th} to 88^{th} aspects, wherein the membrane comprises a hydrophobic micropattern on the internal surface of the wall-contact section or on the flow control valve.

A 90^{th} aspect relates to the device of the 46^{th} to 89^{th} aspects, wherein the membrane is bonded to the braided Nitinol frame with a bonding substrate.

A 91^{st} aspect relates to the device of the 90^{th} aspect, wherein the bonding substrate has a thickness in a range of 5 to 10 microns.

A 92^{nd} aspect relates to the device of the 46^{th} to 91^{st} aspects, wherein the membrane has a thickness in a range of 30 to 50 microns.

A 93^{rd} aspect relates to the device of the 85^{th} to 92^{nd} aspects, wherein the wall contact section of the membrane is bonded to the exterior surface of the wall-contact section of the braided Nitinol frame.

A 94^{th} aspect relates to the device of the 46^{th} to 93^{rd} aspects, wherein the membrane is bonded to the braided Nitinol frame on selected regions of the wall-contact section of the braided Nitinol frame, wherein the selected regions comprise less than 100% of the wall contact section.

A 95^{th} aspect relates to the device of the 94^{th} aspect, wherein the selected regions comprise at least one band around the circumference of the braided Nitinol frame.

A 96^{th} aspect relates to the device of the 94^{th} aspect, wherein the selected regions comprise at least one longitudinal strip on the wall-contact section of the frame.

A 97^{th} aspect relates to the device of the 94^{th} aspect, wherein the selected regions comprise of spots.

A 98^{th} aspect relates to the device of the 46^{th} to 97^{th} aspects, wherein the membrane is made from an elastomer.

A 99^{th} aspect relates to the device of the 46^{th} to 98^{th} aspects, wherein the membrane has a modulus of elasticity in a range of 10 to 20 MPa.

A 100^{th} aspect relates to the device of the 84^{th} to 99^{th} aspects, wherein the flow control valve is a duckbill or Heimlich valve.

A 101^{st} aspect relates to the device of the 84^{th} to 100^{th} aspects, wherein the flow control valve has two lips.

A 102^{nd} aspect relates to the device of the 84^{th} to 101^{st} aspects, wherein the flow control valve opens when air pressure is greater on the distal end than on the proximal end of the device.

A 103^{rd} aspect relates to the device of the 84^{th} to 102^{nd} aspects, wherein the flow control valve controls air to flow predominantly out through the lobar bronchus.

A 104^{th} aspect relates to the device of the 84^{th} to 103^{rd} aspects, wherein the flow control valve has a diameter less than the diameter of the braided Nitinol frame in its expanded state.

A 105^{th} aspect relates to the device of the 84^{th} to 104^{th} aspects, wherein the flow control valve has a diameter in a range of 2.5 to 4.5 mm.

A 106^{th} aspect relates to the device of the 84^{th} to 105^{th} aspects, wherein the flow control valve has a diameter that is 15% to 30% of the diameter of the braided Nitinol frame in its expanded state.

A 107^{th} aspect relates to the device of the 84^{th} to 106^{th} aspects, wherein the flow control valve has a length in a range of 3 mm to 7 mm.

A 108^{th} aspect relates to the device of the 84^{th} to 107^{th} aspects, wherein the flow control valve and the membrane are fabricated together as one component.

A 109^{th} aspect relates to the device of the 45^{th} to 108^{th} aspects, further comprising an airflow resistance adjustment element.

A 110^{th} aspect relates to the device of the 109^{th} aspect, wherein the airflow resistance adjustment element is a tube or rod.

A 111^{th} aspect relates to the device of the 109^{th} or 110^{th} aspects, wherein the airflow resistance adjustment element is biodegradable.

A 112^{th} aspect relates to the device of the 46^{th} to 111^{th} aspects, wherein the wall-contact section has a length in a range of 8 mm to 18 mm.

A 113^{th} aspect relates to an implantable airflow control device for a bronchial passageway comprising: an airflow control valve; a braided wire structural frame expandable from a collapsed configuration to an expanded configuration; and a sealing membrane mounted to at least a distal portion of the structural frame, wherein the sealing membrane forms an enclosed wall defining at least a portion of an airflow passage through the flow control device, and the flow control valve is included in the airflow passage.

A 114^{th} aspect relates to the implantable airflow control device of aspect 113, wherein the airflow control valve is within the braided wire structural frame.

A 115^{th} aspect relates to the implantable airflow control device of aspect 113 or 114, wherein the braided wire structural frame comprises a wall contact section, a first fold, a middle support section radially within the wall contact section and connected to the wall contact section by the first fold, and a second fold connected to the middle support section.

A 116^{th} aspect relates to the implantable airflow control device of aspect 115 wherein the middle support section resides radially within the wall contact section when the airflow control device is in the expanded state and adjacent to the wall contact section when the airflow control device is in the collapsed state.

A 117^{th} aspect relates to the implantable airflow control device of aspects 115 or 116, wherein the middle support section is shorter than the wall contact section.

A 118^{th} aspect relates to the implantable airflow control device of any aspects 113 to 117, further comprising an inner support section connected to the second fold and radially within the middle support section when the device is in the expanded state and adjacent to the middle support section when the device is in the collapsed state.

A 119^{th} aspect relates to the implantable airflow control device of aspect 118, wherein the inner support section is shorter than the middle support section.

A 120^{th} aspect relates to the implantable airflow control device of any aspects 115 to 119, wherein the braided wire structural frame comprises a first braid angle in at least the wall-contact section and a second braid angle in at least the first fold.

A 121^{st} aspect relates to the implantable airflow control device of aspect 120, wherein the first braid angle is less than the second braid angle.

A 122^{nd} aspect relates to the implantable airflow control device of any aspects 115 to 121, wherein the wall-contact section has a length in a range of 8 mm to 18 mm when the device is in the expanded state.

A 123^{rd} aspect relates to the implantable airflow control device of any aspects 113 to 122, wherein the braided wire structural frame has a diameter in a range of 7 mm to 12 mm or in a range of 5 mm to 15 mm or in a range of 11 mm to 14 mm or in a range of 10 mm to 18 mm while the airflow control device is in the expanded state.

A 124^{th} aspect relates to the implantable airflow control device of any aspects 113 to 123, wherein the braided wire structural frame has a length to diameter ratio in a range of 0.28:1 to 0.54 to 1 while in the expanded state.

A 125^{th} aspect relates to the implantable airflow control device of any aspects 113 to 124, wherein the braided wire structural frame has a diameter in the expanded state to a diameter in the collapsed state ration in a range of 4:1 to 7:1, such as about 5.45:1.

A 126^{th} aspect relates to the implantable airflow control device of any aspects 113 to 125, wherein a braided wire forms closed loops at an end of the braided wire structural frame.

A 127^{th} aspect relates to the implantable airflow control device of aspect 126, wherein a plurality of the closed loop ends bend inward toward a central axis of the braided wire structural frame.

A 128^{th} aspect relates to the implantable airflow control device of any aspects 126 or 127, wherein a plurality of the closed loop ends have an angle less than a braid angle of the wall-contact section.

A 129^{th} aspect relates to the implantable airflow control device of any aspects 115 to 128, wherein the first fold has a bend radius in a range of 0.75 mm+/−0.5 mm.

A 130^{th} aspect relates to the implantable airflow control device of any aspects 113 to 129, wherein wires forming the braided wire structural frame form a wall contact section and spokes extending from the wall contact section to a coupler which is releasably attached to the airflow control device.

A 131^{st} aspect relates to the implantable airflow control device of aspect 130 wherein a space is defined between a pair of adjacent ones of the spokes, and the space has an area in a range of 5 mm² to 40 mm².

A 132^{nd} aspect relates to the implantable airflow control device of aspect 130, wherein the spokes each have lengths in a range of 5 to 15 mm.

A 133^{rd} aspect relates to the implantable airflow control device of any aspects 113 to 132, wherein the braided wire structural frame includes a wall contact section and the sealing-membrane covers an outer surface of the wall contact section.

A 134^{th} aspect relates to the implantable airflow control device of aspect 133, wherein the wall-contact section has a length in a range of 8 mm to 18 mm.

A 135^{th} aspect relates to the implantable airflow control device of any aspects 133 to 134, wherein the sealing membrane further comprises a lumen occluding section between the wall-contact section and the airflow control valve.

A 136^{th} aspect relates to the implantable airflow control device of any aspects 122 to 135, wherein the membrane comprises a hydrophilic micropattern surface at least on the wall-contact section or the airflow control valve.

A 137^{th} aspect relates to a flow control device for a bronchial passageway comprising: a flow control valve; a structural frame, wherein the structural frame is expandable from a collapsed configuration to an expanded configuration; a sealing membrane mounted to the structural frame, wherein the sealing membrane forms an enclosed wall defining at least a portion of an airflow passage through the flow control device, and at least one anti-inversion feature within the flow control valve.

A 138^{th} aspect relates to the flow control device of aspect 137, wherein the anti-inversion feature is a at least one joint, such as a weld, between opposite lips of the flow control valve.

A 139^{th} aspect relates to the flow control device of any aspects 137 or 138, wherein the at least one anti-inversion feature occupies only portion of a width between opposite side edges of the lips of the flow control device.

A 140^{th} aspect relates to the flow control device of any aspects 137 to 139, wherein the at least one anti-inversion feature cumulatively occupies a width in a range of 5% to 25% of a width between opposite side edges of the lips of the flow control device.

A 141^{st} aspect relates to the flow control device of any aspects 137 to 140, wherein each of the at least one anti-inversion feature has a width in a range of 0.3 to 1.5 mm and the flow control valve has a width in a range of 7 mm to 10 mm.

A 142^{nd} aspect relates to the flow control device of any aspects 137 to 141, wherein the at least one anti-inversion feature joins opposite lips of the flow control valve at the distal ends of the lips.

A 143^{rd} aspect relates to the flow control device of any aspects 137 to 142, wherein the at least one anti-inversion is in a range of 0.5 to 3 mm from a plane of an inlet to the flow control valve.

A 144^{th} aspect relates to the flow control device of any aspects 137 to 143, wherein the flow control device includes opposing lips, and an inside surface of at least one of the lips is dimpled in a region proximate to the at least one anti-inversion feature.

A 145^{th} aspect relates to the flow control device of any aspects 137 to 144, wherein the wall contact section, in the expanded configuration, is oval in cross-section, and the flow control valve is elongated in cross-section.

A 146^{th} aspect relates to the flow control device of aspect 145, wherein major axes are parallel of the cross-section of the wall contact section and of the flow control valve.

A 147^{th} aspect relates to the flow control device of any aspects 145 or 146, wherein semi-major axes are parallel of the cross-section of the wall contact section and of the flow control valve.

A 148^{th} aspect relates to the flow control device of any aspects 145 to 147, wherein opposite lips of the flow control valve are aligned with the major axis.

A 149^{th} aspect relates to the flow control device of any aspects 145 to 148, wherein the flow control device parts along a line parallel to the major axis.

A 150^{th} aspect relates to the flow control device of any aspects 145 to 149, wherein a surface area of a portion of the sealing membrane spanning a distal end of the braided wire structural frame is in a range of 5% to 15% greater than an area circumscribed by the distal end of the braided wire structural frame in the expanded configuration.

A 151^{st} aspect relates to the flow control device of any aspects 137 to 150, wherein a ring surrounds the flow control valve and the ring is integral with the sealing membrane.

A 152^{nd} aspect relates to the flow control device of aspects 151, wherein the ring has a stiffness greater than a stiffness of the sealing membrane radially outward of the ring.

A 153^{rd} aspect relates to the flow control device of aspects 151 or 152, wherein the ring has a thickness greater than a thickness of the sealing membrane radially outward of the ring

### BRIEF DESCRIPTION OF THE DRAWINGS

FIG. 1 is a schematic illustration of a patient's lungs and airways with the right middle lobe omitted.

FIG. 2A is a schematic illustration of a lobar valve in an unconstrained expanded state.

FIG. 2B is a schematic illustration of a lobar valve in an constrained delivery state.

FIG. 2C is a schematic illustration of a lobar valve implanted in a right upper lobar bronchus.

FIGS. 3A and 3B are schematic illustrations of closed loop ends of a braided structural frame.

FIG. 4 is a schematic illustration of a lobar valve coupler

FIGS. 5A and 5B are schematic illustrations of a lobar valve having inverted spokes.

FIGS. 6A and 6B are schematic illustrations of a lobar valve with spokes made from separate wire loops.

FIG. 7A is a schematic illustration of a lobar valve in a constrained delivery state having undeployed retention barbs.

FIG. 7B is a schematic illustration of a lobar valve in an unconstrained state having deployed radially protruding retention barbs.

FIG. 8 is a schematic illustration of a lobar valve having a braided tubular structural frame forming an airway contact region and a valve housing.

FIG. 9A is a schematic illustration of a lobar valve having a structural frame with both ends open.

FIG. 9B is a schematic illustration of a structural frame with both ends open wherein the ends are bent inward.

FIG. 10 is a schematic illustration of a lobar valve having spokes on both proximal and distal ends.

FIG. 11 is a schematic illustration of a lobar valve formed from a tubular braided structural frame folded inward on itself.

FIG. 12 is a schematic illustration of a delivery tool holding a lobar valve in a bronchoscope.

FIG. 13A is a schematic cross-sectional illustration of a lobar valve having a folding braided Nitinol frame in its expanded state.

FIG. 13B is a schematic illustration of a lobar valve having a folding braided Nitinol frame in its expanded state.

FIG. 13C is a schematic illustration of a lobar valve having a folding braided Nitinol frame in its collapsed state.

FIG. 14 is a schematic cross-sectional illustration of a lobar valve having a folding braided Nitinol frame in its expanded state and having a locking ring anchored distal to a cartilage ring in a lobar bronchus.

FIG. 15A is a schematic cross-sectional illustration of a lobar valve having a folding braided Nitinol frame in its expanded state with a flow control valve having an anti-inversion feature.

FIG. 15B shows the anti-inversion feature of FIG. 15 from a different angle.

FIG. 15C is a schematic cross-sectional illustration of a lobar valve having a folding braided Nitinol frame in its expanded state with a flow control valve having an anti-inversion feature.

FIG. 15D shows another embodiment of an anti-inversion feature.

FIG. 16 illustrates an alignment of a flow control valve of a lobar valve in an oval-shaped bronchus.

FIG. 17 illustrates an alignment of a lobar valve in an oval-shaped bronchus.

### DETAILED DESCRIPTION

The disclosure herein is related to systems, devices, and methods for modifying air flow to and from a targeted portion of a patient's lung, which may be substantially diseased, with an implantable device in order to reduce the volume of trapped air in the targeted portion of lung, thereby increasing the elastic recoil of the remaining lung volume.

The authors/inventors conceived of and disclose herein, implantable lung volume reducing devices and medical techniques for implanting lung volume reduction devices through the trachea and bronchi, using minimally-invasive deployment, bronchoscopic and surgical techniques. The device may be embodied as an endobronchial valve. In some embodiments the endobronchial valve may be intended for implant in a lobar bronchus and is referred to as a lobar valve or lobar flow control valve.

Also disclosed is a novel treatment for patients suffering from hyper-inflated lung (e.g., emphysema, COPD, bronchitis, asthma) comprising the application of a minimally invasive bronchoscopy technique to implant a lung volume reduction device into a lung airway of a patient. The implantable lung volume reduction devices, which may be generally referred to as “lobar valves” disclosed herein are intended to be placed in an airway trunk of a lobe such that a single valve regulates air flow to or from the complete lobe, which may have benefits over previously attempted valves that were intended for multiple valve placement in higher generation airways. Benefits of a lobar valve may include lower cost, faster procedure, easier implantation, easier removal, less risk of pneumothorax due to slower lobe volume reduction, and stronger retention. However, some features of devices disclosed herein may be novel and useful for use in higher generation airways and are not limited to devices configured for placement in a trunk of a lobe.

Anatomy and Design Inputs and Challenges:

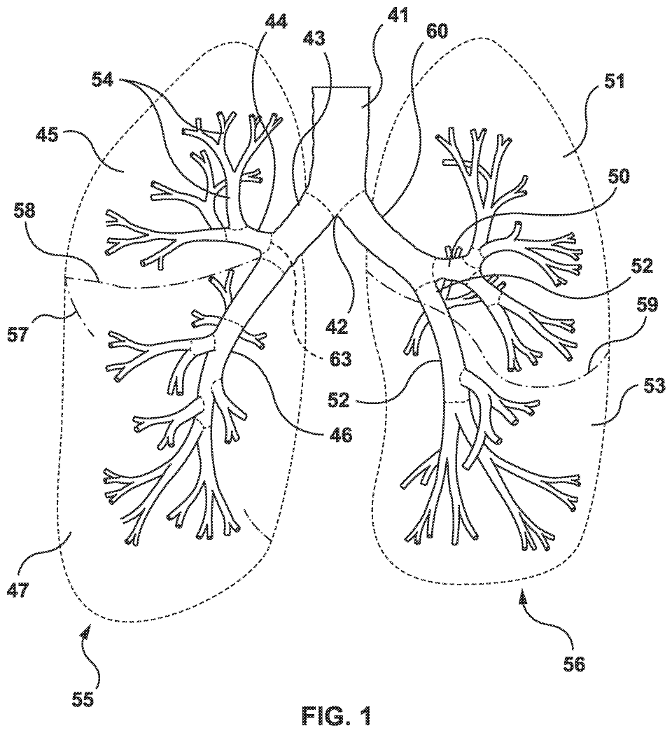

FIG. 1 is a schematic illustration of some anatomical features of human lungs. Air passes through the trachea 41, which divides 42 into the right and left main or primary bronchi 43 and 60. The lungs normally have clear anatomical divisions known as lobes. The right lung 55 is divided into three lobes called superior 45, middle (not shown for simplicity) and inferior 47 lobes, by the oblique 57 and horizontal 58 fissures that are folds of the visceral pleura. The left lung 56, which is slightly smaller, is divided into a superior 51 and inferior 53 lobe, by an oblique fissure 59. The term “proximal direction” refers to the direction along an airway path that points toward the patient's mouth or nose and away from the patient's lungs. In other words, the proximal direction is generally the same as the expiration direction when the patient breathes. The term “proximal section” or “proximal end” of a device implanted in a patient's airway refers to the section or end of the device intended to face the proximal direction. The term “distal direction” refers to the direction along an airway path that points toward the patient's lung and away from the mouth or nose. The distal direction is generally the same as the inhalation or inspiratory direction when the patient breathes. The term “distal section” or “distal end” of a device implanted in a patient's airway refers to the section or end of the device intended to face the distal direction.

Lobar valves may be implanted in a secondary bronchus, also known as a lobar bronchus. Humans have one lobar bronchus providing air passage to each lobe of the lung, including three in the right lung and two in the left lung. The right-side lobar bronchi include the right upper lobar bronchus 44, right middle lobar bronchus (not shown for simplicity), and right lower lobar bronchus 46. The left side lobar bronchi include the left upper lobar bronchus 50 and left lower lobar bronchus 52, both of which divide into tertiary bronchi 54. Overlapping cartilage plates of the lobar bronchi provide structural strength to maintain patency of these bronchi. Typically, a lobar bronchus has a protruding cartilage ring 63 near the proximal end of the lobar bronchus. Humans may typically have lobar bronchi having an average diameter in a range of 6 mm to 18 mm. The average length is about 19 mm (e.g., in a range of about 8 to 40 mm).

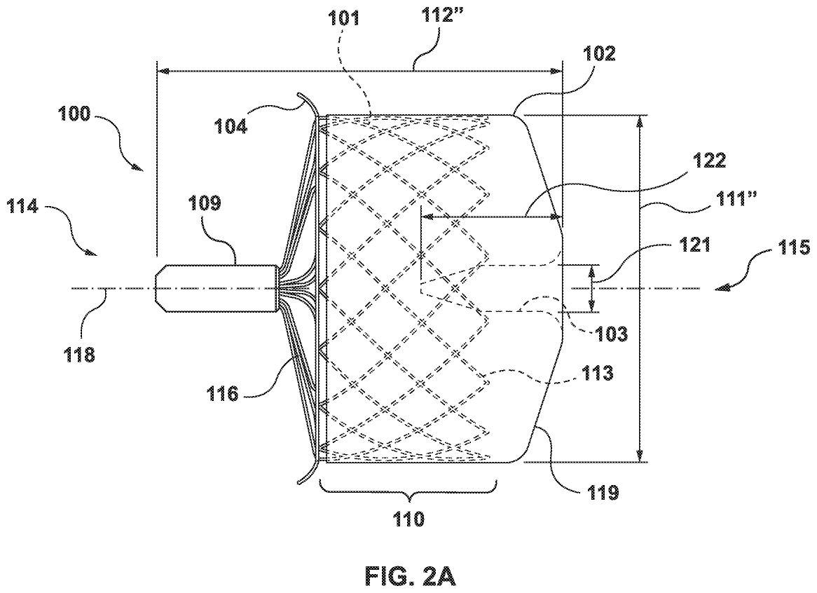

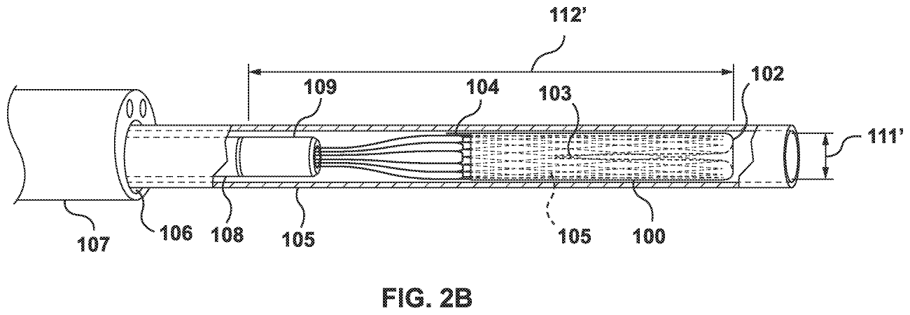

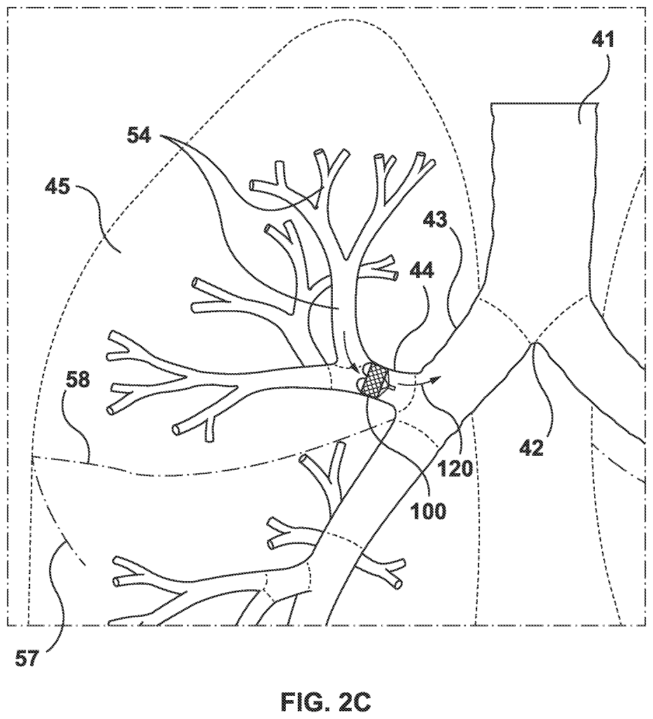

Lobar valves disclosed herein are transitionable from a contracted delivery state to an expanded deployed state. In the contracted delivery state the lobar valve is compressed and constrained in a delivery sheath that can be advanced through a bronchoscope working channel. When advanced out of the delivery sheath the lobar valve transitions toward its expanded state, for example via elastic properties of a structural frame, until constrained by the brochus in which it is implanted. The circumference of the lobar valve in its unconstrained, expanded state may be larger than the circumference of the targeted airway where it is implanted, so that a radial force is applied by the lobar valve to the airway wall. FIG. 2A is a schematic illustration showing general features of a lobar valve 100 in an unconstrained, expanded deployed state having a proximal end 114 and a distal end 115, where the distal end is intended to be implanted deeper into the lung than the proximal end. The lobar valve 100 generally comprises a structural frame 101, a sealing membrane 102, a flow control valve 103, and optionally one or more retention elements 104, such as barbs. The sealing membrane may be connected to the structural frame to function at least in part as an airway seal or an air flow control valve or an anchoring feature. The flow control valve may be part of the sealing membrane or a separate component and functions to allow fluid (e.g., air) to flow at least predominantly out of the targeted lung lobe and restrict flow into the lobe. The retention element may comprise radially extending barbs or other elements that function to hold the device in the targeted airway when exposed to forces such as lung movement and air pressure changes (e.g., coughing, sneezing, breathing).

FIG. 2B shows general features of the lobar valve 100 in a contracted delivery state where it is contained in a delivery sheath 105 that is advanced out of a distal end of a working channel 106 of a bronchoscope 107. A distal end of a delivery tool 108, e.g. extendible shaft, is temporarily attached to a coupler 109 of the lobar valve. Various embodiments of these features may be mixed and matched and lobar valve embodiments are not limited to the combination of these elements presented in the figures.

A lobar valve 100 may assume its contracted delivery state when delivered through a working channel of a bronchoscope, optionally contained in a delivery sheath and manipulated with a delivery tool. The lobar valve and optional delivery sheath and delivery tool may be sized to pass freely through a working channel of a bronchoscope. For example, a lobar valve adapted to be delivered with a delivery tool through a working channel with a 2.8 mm lumen may have a maximum diameter of 2.6 mm (e.g., a maximum diameter of 2.5, 2.4, 2.3, 2.2. 2.1 mm). In some embodiments lobar valves may comprise a structural frame having a delivery state and deployed state, wherein the delivery state has a maximum diameter in a range of 2 (0.0787″) to 2.5 mm (0.0984″), preferably 2.11 mm (0.083″).

Ease of use and procedural expediency is a desired requirement. The lobar valve may be designed to be consistently delivered to a correct location with average physician skill. Compared to valves that are implanted at higher generation airways implanting a lobar valve may be a faster procedure because only one valve needs to be implanted to affect an entire lobe, the lobar bronchi are larger, more proximal and hence easier to access and find than distal higher generation bronchi. Also, assessing the function of a single implanted lobar valve is faster and easier compared to assessing multiple distally implanted valves.

A lobar valve and procedure for implanting one may cost less compared to implanting multiple higher generation valves in particular since there is only one device to implant and the procedure is faster.

Design considerations may also consider particular challenges for placement in a lobar bronchus. For example, the length of a lobar bronchi is relatively short, the length to diameter ratio is considerably smaller, the cross section of a lobar bronchus is radially asymmetrical (e.g., ovular or irregular), and the diameter of the lumen is inconsistent along the length of the lobar bronchus (e.g., flared at the proximal, distal or both ends). Potentially, a single lobar valve placed in a lobar bronchus may experience a greater air pressure difference between its proximal and distal sides compared to a plurality of valves positioned in several higher generation bronchi of a lobe.

Furthermore, each particular lobar bronchus in a patient has unique characteristics such as the angle of approach and geometry.

Structural Frames:

The structural frame provides a framework to hold the membrane and valve in a desired orientation and position in a target bronchus. The structural frame applies an outward radial force to press the membrane against the airway wall and hold the flow control valve in the airway so air is directed through the flow control valve.

The structural frame 101 may be made by braiding wires into at least a generally cylindrical shape. The generally cylindrical shape of the structural frame can constitute an airway wall contact region 110 that is intended to expand to contact the airway wall and to flexibly conform to the surface of the airway wall. The wires may be elastically or super-elastically flexible with shape memory ability, for example the wires may be made from Nitinol that is superelastic above a temperature of body temperature (about 37° C.) or lower. As the braided wire structural frame transitions from the delivery state to deployed state the device diameter (excluding optional radially extending barbs) increases from a first device diameter 111′ (FIG. 2B) toward an unconstrained second diameter 111″ (FIG. 2A); and the device length decreases from a first device length 112′ to a second device length 112″. For example, the first device diameter 111′ may be in a range of 2 mm to 2.6 mm and the second device diameter 111″ may be in a range of 10 mm to 18 mm; the first length 112′ may be in a range of 30 to 40 mm and the second device length 112″ may be in a range of 8 mm to 18 mm.

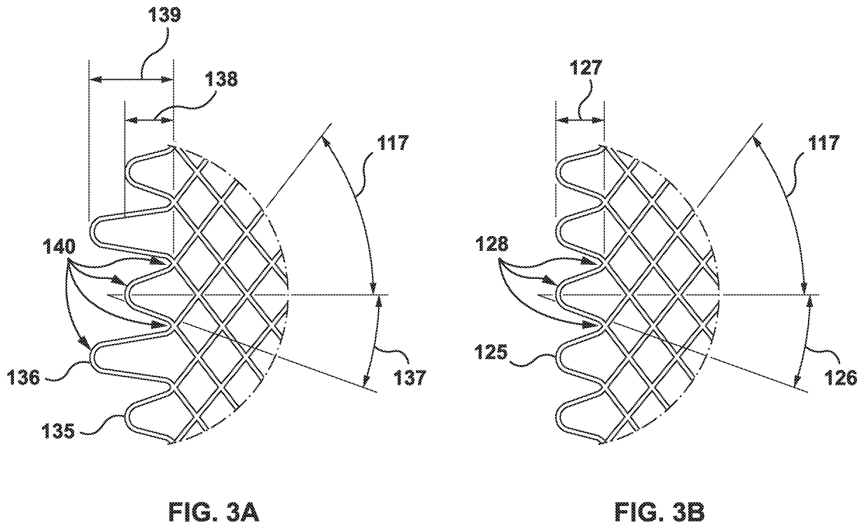

The wires used to form the structural frame braid may be for example superelastic Nitinol wires having wire diameter in a range of 0.003″ to 0.008″ (preferably in a range of 0.005″ to 0.006″). The structural frame 101 may have a braid angle 117 in a range of 35° to 55° (see FIG. 3A). Various embodiments of braid configurations may be used without diverging from the intent of the disclosure.

In some embodiments the wires are braided with a closed loop 113 at the distal end 115 of the device as shown in FIG. 2A. For example, the structural frame 101 may have 48 wires braided with 24 closed loop ends 113 on the distal end 115 of the device and the wire terminals may gathered and shape set into spokes 116 toward the proximal end 114 and fixed into a coupler 109. Optionally, the closed loop ends may be adapted to facilitate collapse of the device 100 into its contracted delivery state. For example, as shown in FIG. 3B the closed loop ends 125 may have a smaller angle 126 (e.g., 22°) than the braid angle 117, which may require less force to bend away from its shape set configuration toward a collapsed configuration. To create a smaller angle 126 the length 127 may extended (e.g., about 2 mm) and have three points of inflection 128 (e.g., having a radius of curvature of about 0.25 mm). In another example as shown in FIG. 3A the closed loop ends may include two or more alternating closed loop end shapes such as a first closed loop end 135 and a second closed loop end 136, which may further facilitate collapse of the device by allowing the first and second closed loop ends to disperse material as they overlap in the collapsed configuration. For example, the first closed loop end 135 may be shorter than the second closed loop end 136 (e.g., the first 135 may have a length 138 of about 2 mm and the second may have a length 139 of about 3.5 mm). Both the first and second closed loop ends 135, 136 may have a reduced angle 137 as compared to the braid angle 117. To create the reduced angles 137 the wires may have three points of inflection 140 (e.g., having a radius of curvature of about 0.25 mm).

Optionally, in the unconstrained state the closed loop ends 113 may be bent inward toward the central axis to relieve forces and friction applied by the ends to the airway wall to reduce the risk of irritating the tissue which may cause granulation tissue or injury.

To accommodate lobar bronchi having an average circumference in a range of 22 mm to 44 mm multiple lobar valves may be provided. For example, a large size lobar valve may have a frame with an airway contact section having a diameter in a range of about 15 to 20 mm, preferably about 16 mm (a circumference of 50.24 mm), which may be intended to be placed in lobar bronchi having a circumference in a range of 31 mm to 44 mm; and a smaller sized lobar valve may have a frame with an airway contact section having a diameter in a range of about 11 mm to 13 mm, preferably about 12 mm (a circumference of 37.7 mm), which may be intended to be placed in lobar bronchi having a circumference in a range of 22 mm to 33 mm. Note that the lobar valves may generally have a maximum unconstrained circumference that is larger than the circumference of the intended lobar bronchus (e.g., about 2 to 2.5 mm larger, about 10 to 20% larger) so that when constrained by the lobar bronchus the airway contact section of the frame firmly contacts the airway wall and applies an outward radial force against the airway wall via the elastic properties of the structural frame and optionally other features described herein that contribute to radial contact force. The target airway may be measured using CT or other medical imaging or with a sizing device delivered through a bronchoscope.

The ratio of the maximum outer diameter of the airway contact section in an unconstrained state to the maximum diameter of the constrained delivery state may be in a range of 3.8:1 to 7.8:1. Due to the relatively larger diameter and short length of lobar bronchi compared to higher generation airways, lobar valves may have a smaller length to diameter ratio in an expanded unconstrained state than current devices intended for more distal positioning. For example, a lobar valve may have a length in a range of 4 mm to 6 mm in its unconstrained state and a length to diameter ratio in a range of 0.545 to 0.286.

The structural frame along with the connected sealing membrane(s) in the delivery state may have a maximum diameter less than 2.7 mm (e.g., less than 2.6, 2.5, 2.4, 2.3, 2.2, 2.1 mm), preferably a maximum diameter of about 2.3 mm. Alternative embodiments of lobar valves may have different dimensions to allow them to be delivered through bronchoscope working channels having different diameters. Optionally, lobar valves in an unconstrained state may have a noncircular cross-section (e.g., ovoid, oval, irregular), which may have an improved fit in a bronchus having a noncircular cross-section. Alternatively, a lobar valve may be adapted to conform to a noncircular airway cross-section or irregular airway wall surface.

In situ, the structural frame may expand and contract with movement of the bronchus (e.g., during elastic recoil). The shape of the structural frame or use of its retention element may be resistant to tilting or may function properly when positioned in a range of angles with respect to the axis of the bronchus. Also, the structural frame may be compressed after it has been fully deployed allowing for repositioning. For example, a structural frame may be compressed by grasping or coupling a delivery tool to the frame's coupler and at least partially withdrawing it into a delivery sheath.

In its contracted delivery state, for example as shown in FIG. 2B, a structural frame 101 including its optional spokes 116 and coupler 502 may be sufficiently flexible to pass through a lumen 106 of an endoscope 107 (e.g., bronchoscope) when the endoscope is bent to traverse a tortuous airway (e.g., having a radius of curvature as small as 15 mm).

Optionally or alternatively, a structural frame may be made from a bioresorbable material such as a polymer matrix (e.g., PLA, PLAGA, PDLLA).

Optionally or alternatively, a structural frame may be balloon expandable or made from a plastically deformable material such as plastic, cobalt chrome alloy, martensitic Nitinol, stainless steel, silicone or urethane.

Optionally or alternatively, a structural frame may be impregnated with an agent such as an antifungal, antibacterial, antimitotic, or anti-inflammatory agent that may improve patient response to implanting the device.

In these embodiments, the wall contact region 101 may be adapted to comply to lobar bronchi that have oval or irregular lumen cross sections; the device may comply to irregular airway surfaces creating a seal on surfaces having bumps, ridges, grooves or other non-smooth surface; the device may have an overall length that is suited for fitting in lobar bronchi.

The wall contact area 110 may have flexibility and elasticity to conform to non-cylindrical (e.g., irregular, oval, tapered, flared) or non-smooth (e.g., bumpy, ridged, contoured) airways or alternatively apply a greater contact force that causes the airway wall to deform or a combination of both in order to provide a continuous circumferential sealing band to prevent air leakage in to a targeted portion of the lung under pressure differentials normally experienced in the lung. When implanted in a target airway, a structural frame may be adapted to impart an outward contact force that may expand the airway wall no more than 20% which is expected to provide strong contact and a good air seal while avoiding trauma to the tissue that otherwise could cause excess formation of granulation tissue.

Optionally, a wall contact area 110 in its unconstrained state may be barrel shaped (e.g., have a wider middle than proximal and distal ends) or be flared (e.g., have a larger diameter distally than proximally), which may facilitate creating a good contact region and seal with the airway wall.

The wall contact region 110 of the structural frame 101 provides a scaffold for the membrane 102, which is affixed to the frame, for example by dip coating, adhesive, solvent bonding or other form of bonding. The structural frame may be collapsible to its contracted delivery state in an orderly fashion that does not damage the membrane.

Spokes

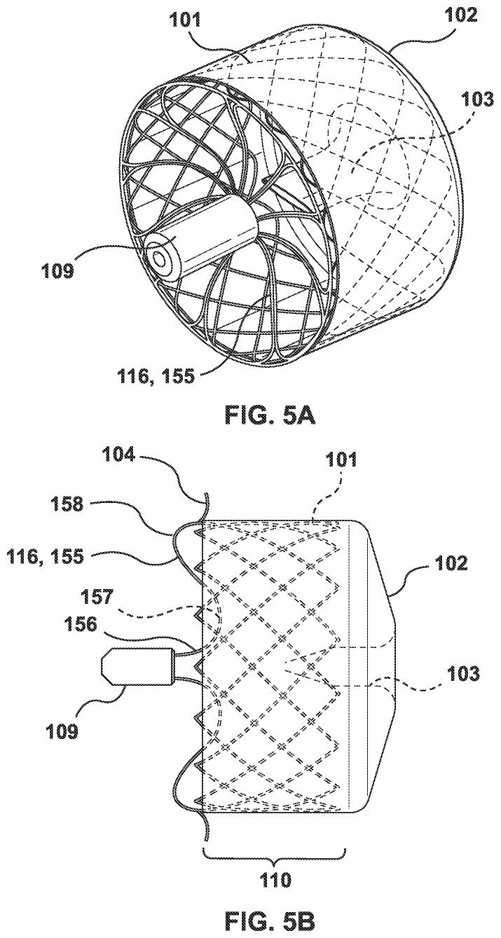

Optionally, a lobar valve may have radial spokes 116 that connect to the airway contact region 110 of the structural frame and extend inward toward the axis 118 where they may be connected to a hub or a coupler 109. In its compressed delivery state (FIG. 2B) the spokes 116 may transfer force (e.g., axially directed push or pull translation or rotation) applied to the coupler 109, for example by a delivery tool 108 attached to the coupler, to the airway contact region 110. The spokes may impart an elastic force radially outward to the airway contact region but shall not apply sufficient force to interrupt the air sealing function of the airway contact region. When a device 100 is in its expanded state and a delivery sheath 105 is advanced over the coupler 109 the force applied by the delivery sheath to the spokes 116 may cause the spokes to radially contract and collapse the airway contact region 110 allowing the device to be pulled back into the delivery sheath or to at least partially reduce the diameter of the airway contact region. This may be used to remove contact force with the airway wall to facilitate repositioning of the device. Optionally, spokes 116, 155 may have a proximal take-off section 156 (FIG. 5B) that is shape set with a concave curve or lesser angle to the coupler than the rest of the spokes which may facilitate collapse of the device by advancing a delivery sheath which will first apply force to the take-off section to begin collapsing the spokes. In some embodiments, as shown in FIGS. 5A and 5B, spokes 155 may have an “S” shaped curve that positions the coupler 109 longitudinally closer to the wall contact region 110 thereby reducing to overall length 112″ of the device when it is in its expanded state. The “S” shaped spokes 155 have a first inflection 157 and a second inflection 158. The “S” shaped curve of the spokes can impart a greater radial force between the device and airway wall, which improves retention of the device in a desired position. While implanting a device having “S” shaped spokes the device may first expand into contact with the airway wall when the sheath 105 is retracted allowing the structural frame to elastically transform to shape set configuration. Then a slight push of the delivery tool 108 may move the coupler 109 distally while the airway contact region 110 remains in place due to radial force and optionally other retention features. This can cause the “S” shaped spokes to impart an increased radial force then a decreased radial force as the coupler moves longitudinally along the axis 118 to its resting position facilitating retention and creating a haptic snap fit that can confirm the device is implanted firmly and with a proper fit.

Coupler

The proximal end of the structural frame may comprise a coupler that mates with a delivery device that allows the coupler to transmit rotational and translational force from the delivery tool to the structural frame. The coupler may be used as a graspable protrusion to grasp with a bronchoscopic tool to manipulate the device during implantation, repositioning, or removal.

For example, a lobar valve 100 may optionally have a coupler 109, positioned at the proximal end 114 of the device, that functions to mate with a coupler of a delivery shaft 108 and release from the coupler of the delivery shaft upon actuation by a user. For example, the coupler may have a geometry (e.g., male or female threading) that mates with a coupler of the delivery shaft 108. An actuator (e.g., rotary dial, trigger, slider, button) controllable by a user for example on a handle connected to the delivery sheath and delivery shaft may control the delivery shaft and sheath to control release of the couplers (e.g., retract the sheath 105 and rotate the delivery shaft 108 to unscrew the mating coupler). When attached the coupler transmits motion of the delivery shaft to the implantable valve 100 including longitudinal translation distally, proximally and rotation around longitudinal axis 118.

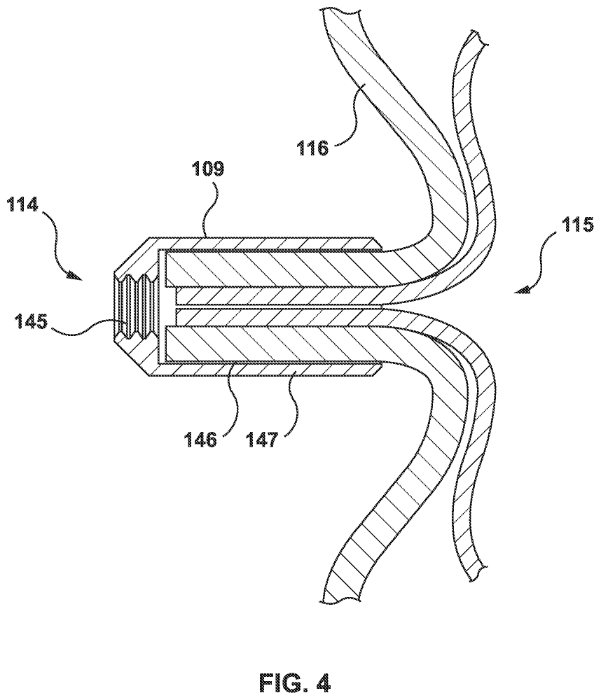

In embodiments having spokes 116 a coupler 109 may also function to contain the terminals of the spokes. FIG. 4 shows a coupler 109 that is a rigid tube having a female threaded section 145 on its proximal end 114 for mating with a male threaded coupler on a delivery tool 108 (FIG. 2B). A lumen 146 defined by the walls of the rigid tube 147 hold ends of the spokes 116, which may be for example terminals of wires used to braid the structural frame 101 (FIG. 2A) or alternative spoke elements.

Optionally, a coupler may be laser cut from a Nitinol hypotube, which may also form spokes and radially protruding retention barbs.

A coupler may have a length in a range of 1 to 4 mm (e.g., about 3 mm)

Covering/seal/membrane

Lobar valves disclosed herein may further have at least one membrane (102 in FIG. 2A) connected to the structural frame 101 that functions to create an air seal of the lobar bronchus permitting air to flow only or at least predominantly through a flow control valve 103. The material of the sealing membrane 102 may further function to resist tissue ingrowth so the lobar valve may be safely removed after a prolonged period of remaining implanted. The material may be made from a material or have a layer that avoids it from sticking to itself, which facilitates transformation of the lobar valve from a collapsed delivery state to an expanded deployed state.

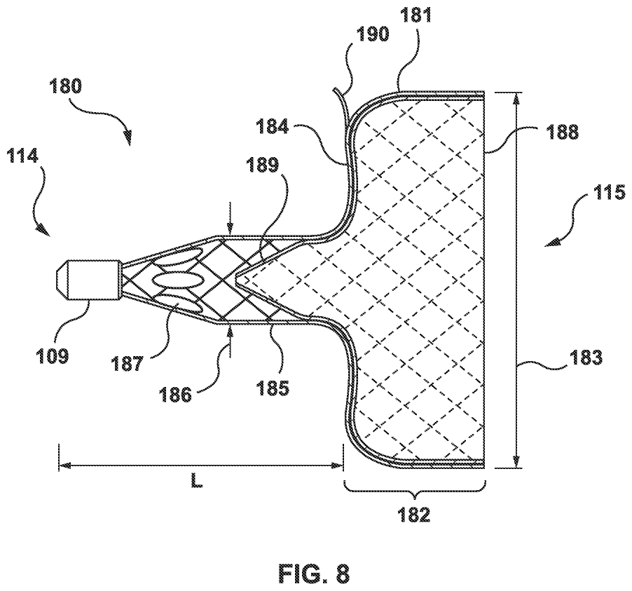

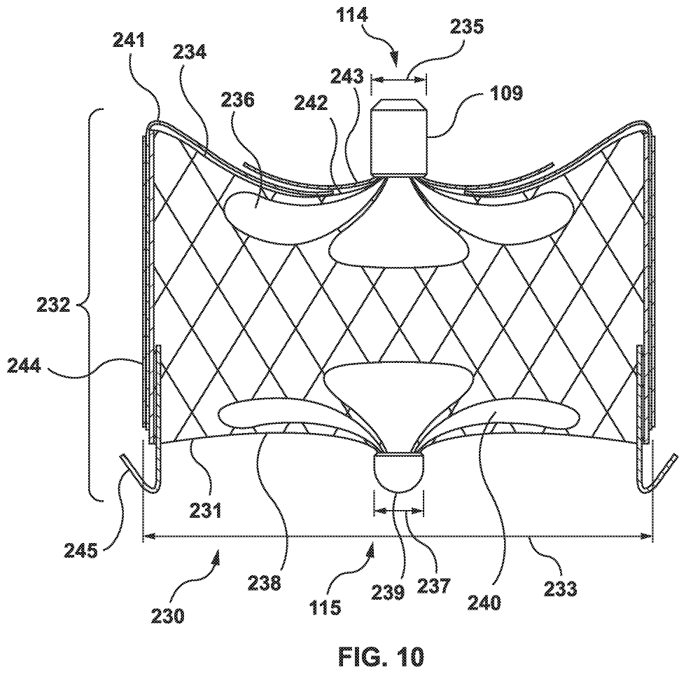

The membrane connected to the structural frame may be made from a thin, flexible, durable, foldable, optionally elastic material such as urethane, polyurethane, ePTFE, silicone, Parylene, Elast-eon™ or a blend of multiple materials. Durometer of the membrane material may be in a range of 70 A to 85 A. The membrane may be made by insert molding, dip coating or spray coating a mold or other manufacturing methods know in the art of medical balloon or membrane manufacture. It may be bonded to the frame for example by coating the frame, laminating over the frame, dip coating, spray coating, heat staking, bonding with adhesive, solvent bonding, or sewing. For example, the membrane may have a thickness in a range of 30 to 50 microns thick and may be bonded to the structural frame with an adhesive substrate that has a thickness in a range of 5 to 10 microns, which may provide sufficient bonding strength while allowing sufficient flexibility of the flow control device so it can easily transition between its contracted and expanded states. Referring to FIG. 2A as an example, a membrane 102 may cover the wall contact region 110 of the structural frame 101 and at least a portion of a luminal covering region 119 to disallow air from flow through a lumen of the bronchi except for through the flow control valve 103 and impede air from leaking around the edges between the wall contact region and an airway wall. A luminal covering region 119 may be on the distal side 115 of the wall contact region 110, or in some embodiments on a proximal side 114 (e.g., FIGS. 8 and 10). The luminal covering region 119 may be flat or have a convex shape that bulges out distal to the airway contact region 110 as shown in FIG. 2A. The extra material of a bulging luminal covering region 119 may allow the structural frame to conform to a non-circular airway cross section. The membrane material may also be partially stretchy to allow it to conform to irregular airway geometry. For example, the membrane material may be rated for up to 500% elongation.

The sealing membrane may be positioned and bonded outside the structural frame. Alternatively, a sealing membrane may have an inner membrane layer bonded to the inner surface of the structural frame as well as an outer membrane layer bonded to an outer surface of the structural frame wherein the inner and outer layers may be bonded to one another between braid wires or spokes 116 thus encapsulating at least a portion of the structural frame.

Airflow 120 as shown in FIG. 2C flows from the lobe distal to the device 100, through a valve 103, and out of the lung. The sealing membrane 102 in combination with the flow control valve 103 impedes air from flowing the opposite direction into the lobe. Optionally, the membrane may also form the flow control valve, or alternatively a flow control valve may be a separate structure connected to a structural frame or sealing membrane.

Portions of the sealing membrane 102 framed by wires of the structural frame in the airway contact region 110 may be flexible and have slack that functions to facilitate air sealing by billowing out and applying contact pressure with the airway wall over a surface area defined by the sealing membrane portions when air is passing through the device or a pressure difference is higher within the device.

The sealing membrane 102 and structural frame 101, in particular the wall contact region 110, form a contact surface area that is continuous around a circumference of a targeted airway.

In an alternative embodiment of a sealing membrane the membrane may have channels that intentionally allow air to pass the seal in either direction initially after the device is implanted and gradually close to block air passage except for through a valve. For example, the channels may be positioned on the seal surface next to the airway wall and over time (e.g., a few weeks) become plugged with mucus that naturally exists in the airway. Gradual or delayed sealing could delay the evacuation of trapped air and subsequent lobar volume reduction so that shifting of the lobes of the treated lung occurs more gradually, which may be less likely to have adverse events such as pneumothorax or injury to healthy lung tissue.

Optionally, a membrane may deliver a chemical agent released slowly over time. For example, the membrane may deliver an antiseptic, antimicrobial or other agent, which may reduce the risk of infection, pneumonia, rejection or other complication. For example, a membrane may be impregnated with an agent such as an antifungal, antibacterial, antimitotic, or anti-inflammatory agent that may improve patient response to implanting the device.

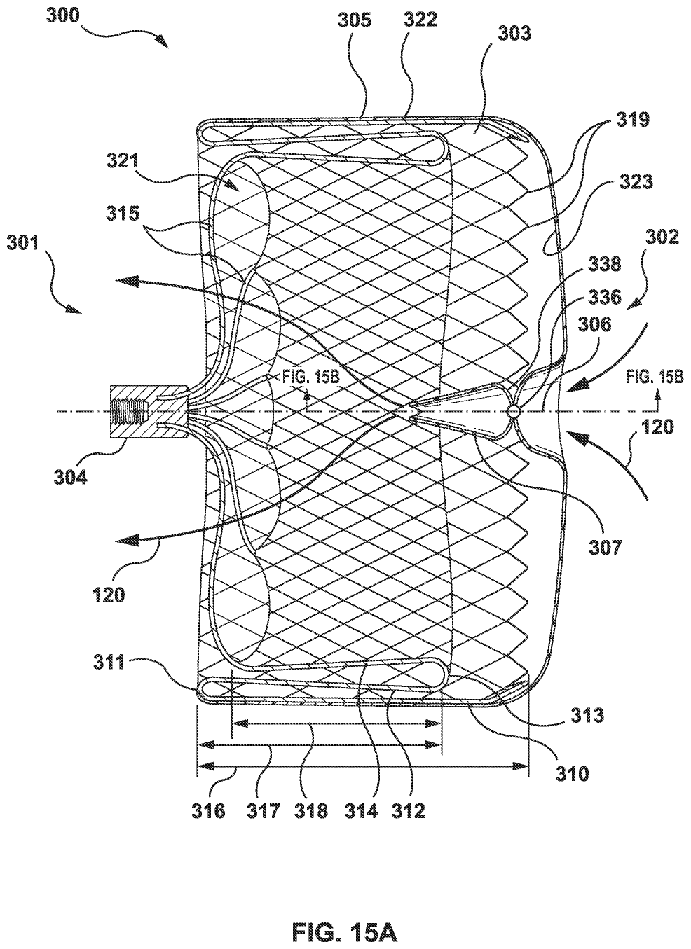

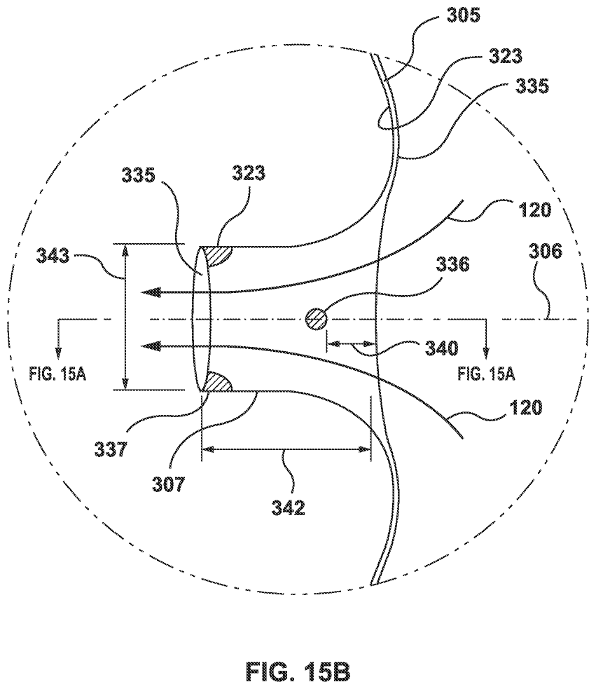

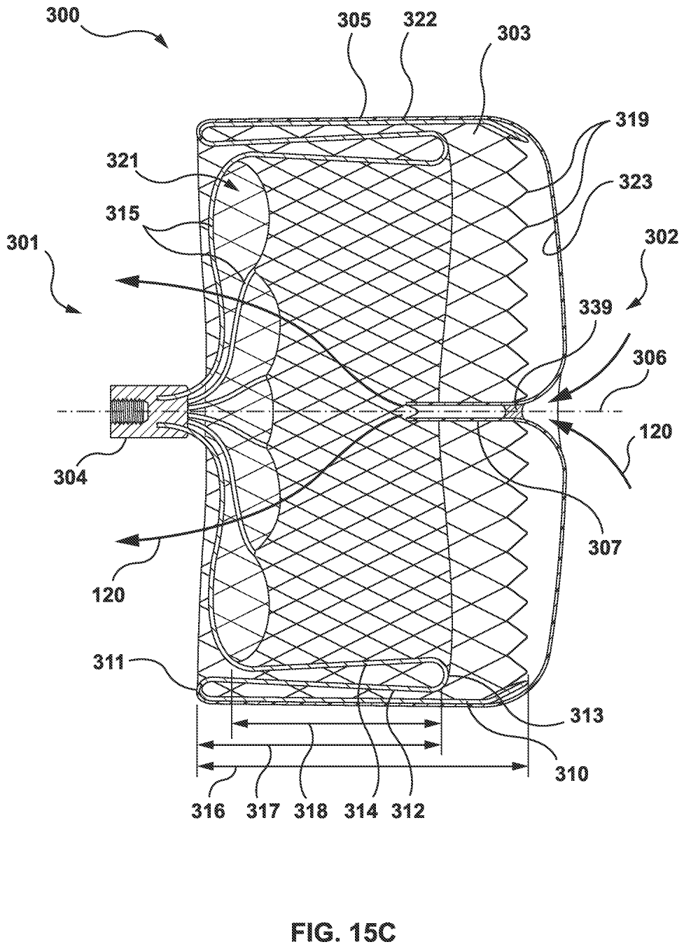

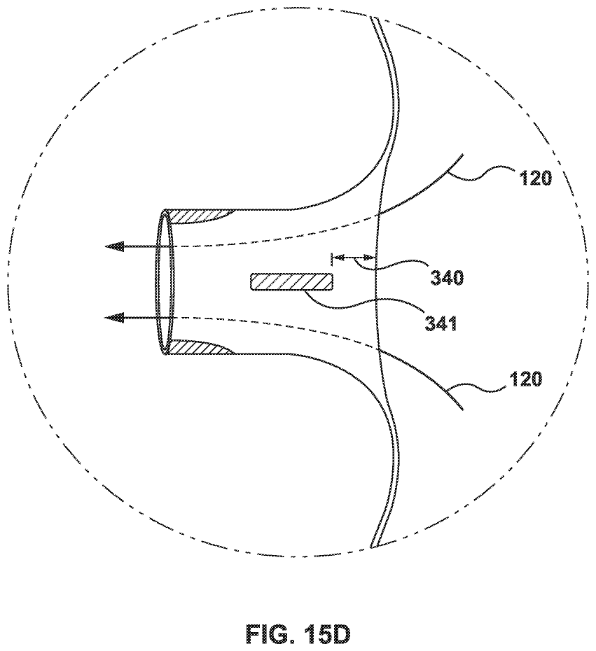

Optionally, a membrane 102 may have a micropatterned surface that provides a non-stick or hydrophobic feature on the interior side (i.e., facing inward toward the axis 118) of the airway contact region 110, on the luminal covering section 119, on the valve, or a combination. The non-stick micropatterned surface may have a lubricious texture pattern which may reduce friction and repel or allow fluids such as mucous to slide off the membrane. As shown in FIG. 15B, a hydrophobic or non-stick surface may be applied on the membrane that forms the inner surface of the valve 335, which may help to reduce surface tension between the sides of the valve reducing the tendency for them to stick together, which in turn may improve function of the valve to release air. Optionally, the hydrophobic surface applied to the inner surface of the valve 335 may continue to at least a portion of the outer surface of the membrane 335 in the luminal covering region 119. The hydrophobic feature of the micropatterned surface may be created by nanostructures molded on to the polymeric membrane 102.

Optionally, a hydrophobic coating may be added to the interior side of the airway contact region 110, on the luminal covering section 119, on the valve, or a combination.

Optionally, a membrane 102 may have a micropatterned surface (e.g., hydrophilic) that provides increased friction on the exterior side of the airway contact region 110.

Flow control Valve

The lobar valve 100 is adapted to provide a seal that does not allow air to flow, or at least substantially increases resistance to airflow through the targeted airway except for through the flow control valve 103. The sealing function is achieved by the membrane 102 connected to the structural frame and the sealing membrane 102 may also form the flow control valve 103. Alternatively, a valve may be a separate structure bonded to the sealing membrane or structural frame. Generally, a valve is adapted to allow air to flow at least predominantly in one direction, from the affected lobe and not into it.

Optionally, a valve material may be impregnated with an agent such as an antifungal, antibacterial, antimitotic, or anti-inflammatory agent that may improve patient response to implanting the device.

As an example, a flow control valve 103 may be made from a flexible, non-stick material such as an elastomeric material, urethane, polyurethane, ePTFE, silicone, Parylene or a blend of multiple materials. The flow control valve 103 may be a duckbill or Heimlich valve having a somewhat funnel shape that transitions from a distal flared, or funnel-shaped end to a proximal closing end. The distal flared end may be tubular having an outer diameter that connects with the luminal covering region 119 of the sealing membrane 102. The distal flared end may have a height 121 in a range of 1 mm to 4 mm (e.g., 2 mm to 3 mm), and a width in a range of 8 to 10 mm. The length 122 of the flow control valve 103 may be in a range of 3 to 8 mm (e.g., 5 mm). The Heimlich valve 103 includes a pair of opposed, inclined walls having ends that meet at lips at the proximal closed end. The lips meet at two opposed corners and may be pinched flat. The walls can move with respect to one another so as to separate at the lips and form an opening through which fluid can travel. When exposed to fluid flow in a direction represented by the arrow 120 in FIG. 2C at a cracking pressure, the walls separate from one another to form the opening through which the fluid may flow. When exposed to fluid flow in an opposite direction the lips remain closed and prevent fluid from flowing through the duckbill valve. Alternatively, other forms of flow control valves known in the art of medical devices may be used. Optionally, the lips may be normally opened at least a small amount when there is no pressure differential across the valve, which may reduce or eliminate the cracking pressure and reduce an opening response time.

Optionally, a flow control valve 103 may be adapted to provide a desired exiting air flow resistance. It may be desired to release air from the target lobe slowly to reduce a risk of pneumothorax that can be caused by rapid deflation of the lobe. Exiting air flow resistance may be inversely proportional to the valve's lumen diameter proportional to its length and may be a function of material stiffness.

Any of the lobar valve embodiments disclosed herein may optionally have an airflow resistance modulation feature that initially and temporary allows some air to flow from the proximal end 114 to the distal end. This feature may function to slow down the volume reduction of the targeted lobe to reduce a risk of pneumothorax associated with rapid deflation. For example, the feature may be a biodegradable or dissolvable component that holds the flow control valve 103 partially open or provides a gap between the airway contact region of a device 100 and the targeted airway wall. The component may shrink or dissolve over an initial duration of time (e.g., in a range of 3 days to 3 weeks). Alternatively, a physician may implant the flow control device with an airflow resistance modulation feature in place and perform follow up procedure, for example within 3 days to 3 weeks following the initial implant procedure to block, remove or reposition the airflow resistance modulation feature, which may increase the resistance to inhalation. The physician may assess the rate of lobar volume decrease or integrity of the pleura following the initial implantation to determine if the airflow resistance should be increased. Furthermore, a physician may elect to decrease inhalation resistance even more if the targeted lobe is found to collapse too aggressively following an initial implant. This may be done in a follow up procedure that adds a different airflow resistance modulation feature that reduces resistance further. An airflow resistance modulation feature may be in a form of a tube positioned in the valve path that restricts how much the valve closes during inhalation either by holding the valve open or by providing a lumen in the tube through which air may flow. Alternatively, instead of a tube a soft rod (e.g., a rod made from a polymeric material such as silicone beading) may be used to prevent the valve from closing completely. Another alternative embodiment of an airflow resistance modulation feature is a hole in the membrane (e.g., positioned in the part of the membrane that covers the lumen of the airway) that may be filled when desired for example by applying an adhesive to fill the hole. Another alternative embodiment of an airflow resistance modulation feature involves a biodegradable suture that partially holds the valve open and allows the valve to fully close when the suture dissolves.

A flow control valve, for example a Heimlich or duckbill valve, optionally may have a feature that prevents the valve from inverting inside out, which otherwise could be caused by a higher pressure on the proximal side of the valve. For example, as shown in FIG. 15A, a flow control valve 307 may have two lips that are tacked together at at least one position between the lips forming an anti-inversion feature 336. The anti-inversion feature 336 may be a weld joining the lips and may occupy only a portion of the space between the lips so the remaining portion between the lips is open to allow air flow 120 in one direction. The weld may be a point, such as in the center between opposite side edges of the lips. The weld may occupy less than 20% to less than 5% the width of the valve, e.g., the distance between opposite edges of the lips.

FIG. 15B shows the flow control valve 307 of FIG. 15A rotated 90 degrees, wherein the anti-inversion feature 336 is shown as a dot in the center of the flow control valve's width and wherein air flow 120 is allowed to pass to each side of the anti-inversion feature 336. Alternatively as shown in FIG. 15D, an anti-inversion feature 341 may elongated and parallel to the axis of the valve, for example in the shape of a stripe, which may increase the strength of the feature without occupying more of the width of the valve's opening. Optionally, the flow control valve may have multiple anti-inversion features in the lumen between the lips. Optionally, the flow control valve may have multiple anti-inversion features 337 at the edges of the flow control valve (see FIG. 15B), preferably at the proximal end of the valve as shown and at the corners of the valve's lips. Anti-inversion features on the edges of the valve may be dots as shown in FIG. 15B or be elongated and optionally tapered as shown in FIG. 15D. Anti-inversion features may have a width in a range of 0.3 to 1.5 mm (e.g., 0.5 mm), which has been found to provide sufficient strength to prevent inversion under expected pressure differences.

Preferably, the remaining open portion of the flow control valve where air may flow has a width in a range of 7 to 10 mm to permit sufficient flow of exhaled air. An anti-inversion feature 336, 341, particularly those positioned in the center of the valve 307 may be positioned at a distance 340 from the distal flared end in a range of 0.5 to 3 mm (e.g., 0.6 to 1 mm).

In the exemplary embodiment shown in FIG. 15A the two lips of the flow control valve 307 are dimpled inward to where the anti-inversion feature 336 connects the two lips. The dimples 338 allow the lips of the valve to remain further apart toward the distal end of the valve. Alternatively, as shown in FIG. 15C, an anti-inversion feature 339 may hold the lips closer together, being absent of dimples.

The anti-inversion features 336, 337, 339, 341 may be formed by heat welding the two layers of membrane that form the flow control valve together. Optionally, this may involve a mandrel for holding the membrane or masking regions where welding is to be avoided. Alternatively, the anti-inversion features may be formed by applying adhesive between the membrane layers in the desired position.

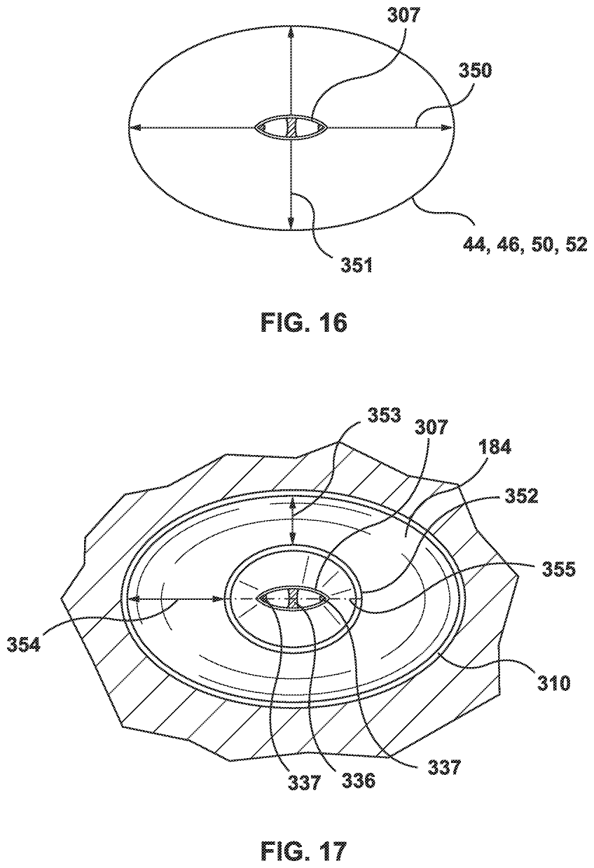

Optionally, the lobar valves disclosed herein may have features that improve function when they are placed in lobar bronchi having oval or irregular transverse cross sections. For example, as shown in FIG. 17, a lobar valve is placed in a lobar bronchus having an oval cross section. The wall contact area 310 conforms to the shape of the bronchus, in part due to the flexible structural frame applying radially outward pressure against the bronchus wall, and the flexible membrane adjusts according to the shape of the structural frame. The luminal covering region of the membrane 184 may be sized to have a little slack when the structural frame is fully expanded in an unconstrained configuration. For example, the surface area of the luminal covering region may be 5% to 15% larger than the cross-sectional area circumscribed by the structural membrane in an unconstrained deployed configuration. Optionally, there may be more slack in a first direction (major axis) 350 than in a second direction (semi-major axis) 351 (see FIG. 16) and the lobar valve may be positioned in an oval-shaped lobar bronchus with the first direction 350 aligned with the semi-major axis of the oval. This slack, optionally in combination with stretchability, may allow the lobar valve to conform to an oval or irregular bronchus without negatively impacting the function of the flow control valve, which is within the luminal covering region 184. Optionally as shown in FIG. 17, a lobar valve may have a ring 352 on the luminal covering region 184 of the membrane that surrounds the flow control valve 307, wherein the ring is less stretchy or flexible than the remainder of the membrane in the luminal covering region. The ring 352 may be formed for example with a thicker portion of membrane (e.g., about twice as thick) formed during a molding process, or by adding material after the membrane has been molded, or by heating the membrane in the position of the ring. The ring may be positioned next to or within about 4 mm of the distal end of the flow control valve to provide sufficient amount of membrane in the luminal covering region 184 but outside of the border formed by the ring, to allow it to deform or stretch while minimizing deformation of the membrane within the ring including the flow control valve 307.

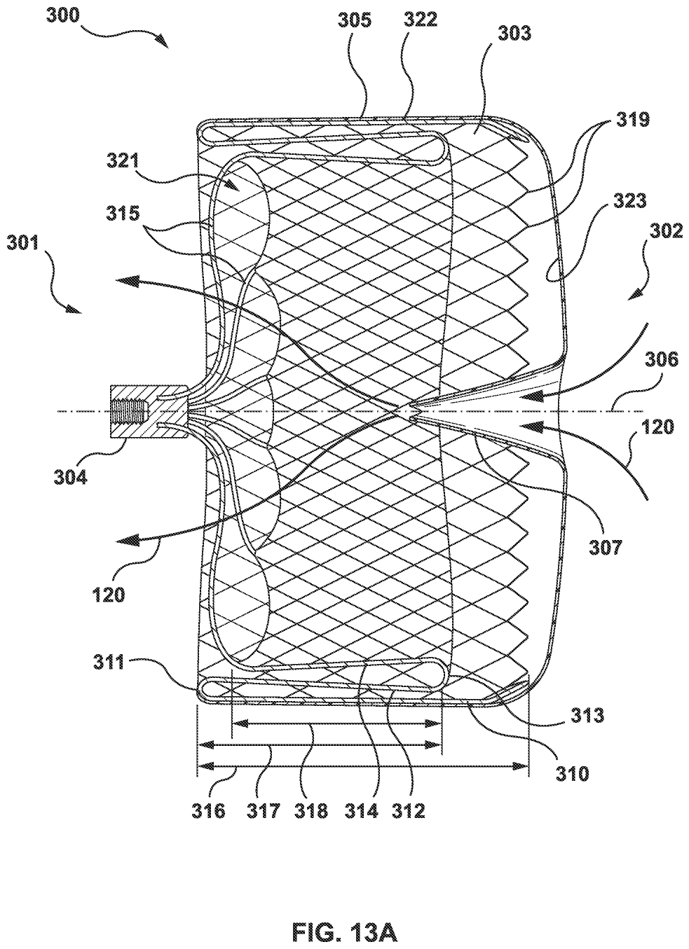

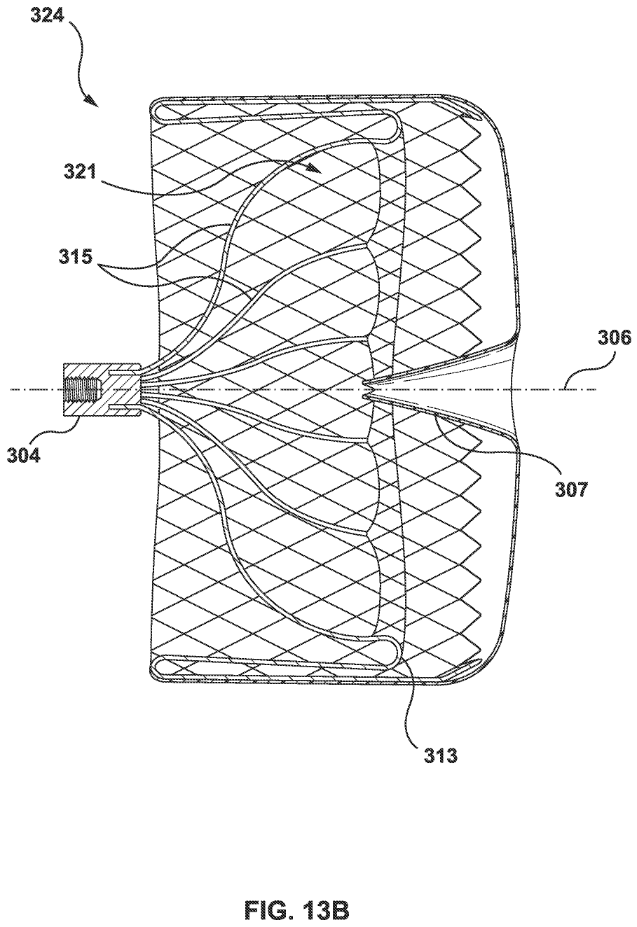

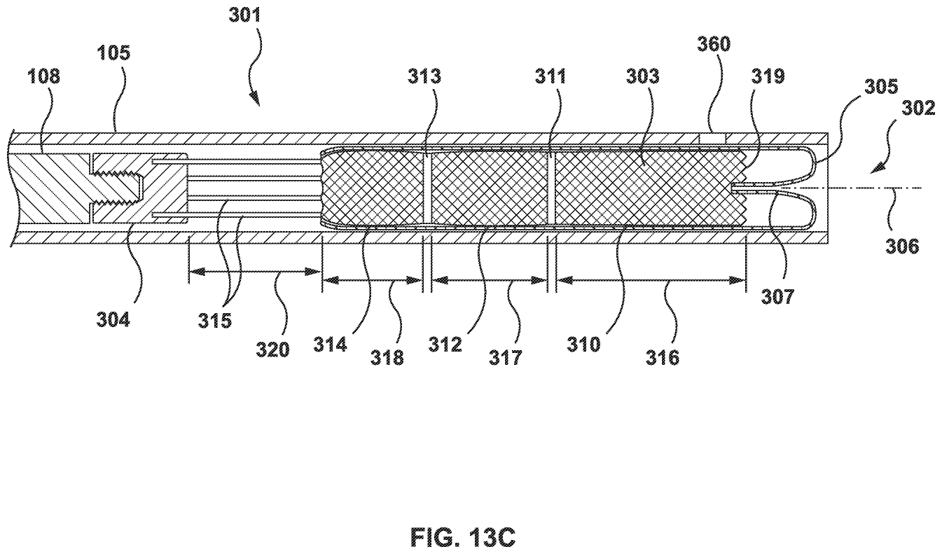

Optionally, a lobar valve may have a structural fame that has an oval transverse cross-sectional shape with a semi-major axis and a semi-minor axis in its unconstrained deployed configuration and may also have a membrane with greater slack in a first direction in the luminal covering region, wherein the semi-major axis is aligned with the first direction. Furthermore, the flow control valve 307 having a lips that part along a parting line/plane 355 between the lips may be aligned so that the parting line is parallel to the major axis 350. This configuration may improve the function of the valve in an oval shaped bronchus. In a scenario where the patient coughs for example an oval-shaped bronchus may be compressed momentarily, typically causing a large reduction in the semi-minor axis of the bronchus. If a flow control valve is aligned so that the parting line 355 is oriented parallel to the semi-major axis of the bronchus as shown in FIG. 17, compression of the semi-minor axis may squeeze the lobar valve in a direction that closes the flow control valve lips. Following the bronchial compression caused by a cough, the lobar bronchus recovers to its normal oval shape and the flow control valve 307 easily opens and its function is not impeded. A delivery device may include a sheath 105 with a visual marker 360 on (see FIG. 13C) its outer surface near its distal end that can be seen when the sheath is extending from a bronchoscope. The visual marker may be oriented with a known orientation of the loaded valve, for example in the direction facing a semi-minor or semi-major axis of the valve or facing the alignment of the parting line 355 of the valve lips. When implanting the lobar valve a user may visually aim the visual marker 360 in a direction that orients the lobar valve properly in the oval shaped bronchus, for example so that the semi-major axis of the valve or the parting line of the lips is aligned with the semi-major axis of the oval shaped bronchus.

Retention Mechanism

A lobar valve may have a retention mechanism such as radial contact force, radially extending barbs, a micropatterned surface on the membrane, placement distal to cartilaginous rings, radial interference, or a combination of these. The retention mechanism functions to keep the device situated and oriented in the targeted position of the patient's airway. The device may be removed by applying force (e.g., pulling, torqueing) to the coupling element or structural frame to overcome the retention force. Alternatively, the retention mechanism may be released from the airway by collapsing the lobar valve.

Radial contact force applied by the airway contact region 110 to the airway wall can help to retain the device 100 in the desired implant location in a lobar bronchus by contributing to friction. Furthermore, radial contact force may distend the airway wall creating a niche for the device to sit in. Radial contact force may be created by the elastic properties of the structural frame 101 returning to its shape set configuration, which may be larger (e.g., 5 to 20% larger) than the airway. Additional radial contact force may be created by optional spokes 116.

FIG. 2A shows radially extending barbs 104 extending from the proximal end 114 of the airway contact region 110. Alternatively, the barbs may extend from the distal end 115 or along the airway contact region 110. The barbs may be made from thin wires connected to the structural frame adapted to protrude beyond the diameter 111″ of the airway contact region 110 when the device 100 is expanded in situ. For example, the barbs may protrude up to 3 mm (e.g., about 1 mm) and be made from wire having a diameter in a range of 0.003″ to 0.008″ (e.g., about 0.005″). The wire may be superelastic Nitinol. Alternatively, barbs may be made from the wires braided to form the structural frame 101. For example, some of the wire terminals may be shape set to become barbs or some of the closed loop ends 113 may be cut and shape set to form barbs. Alternatively, barbs 104 may be made from a laser cut tube, which may also form spokes 116.

A micropatterned surface on the polymeric membrane 102 at least in the airway contact region 110 may help to retain the device in place by resisting sliding on wet surfaces such as airway walls but not on dry surfaces such as through a delivery sheath. For example, a micropattern may be molded to the membrane using techniques known in the art (e.g., U.S. Pat. No. 8,720,047 assigned to Hoowaki, LLC). The micropatterned surface may increase water tension when contacting a wet surface which can greatly increase retention ability. The micropattern may have a plurality of pillars having height and width dimensions less than 1000 nanometers.

Placement of the device just distal to a cartilage ring in an airway may contribute to retention of the device. Cartilage rings exist in lobar bronchi in particular at the proximal end of lobar bronchi and may protrude from the airway surface where cartilage rings are absent. Since the structural frame is shape set to a larger size than the airway it elastically expands against the airway wall. To overcome the cartilage ring, the structural frame would have to reduce in size which goes against its elastically expanding nature.

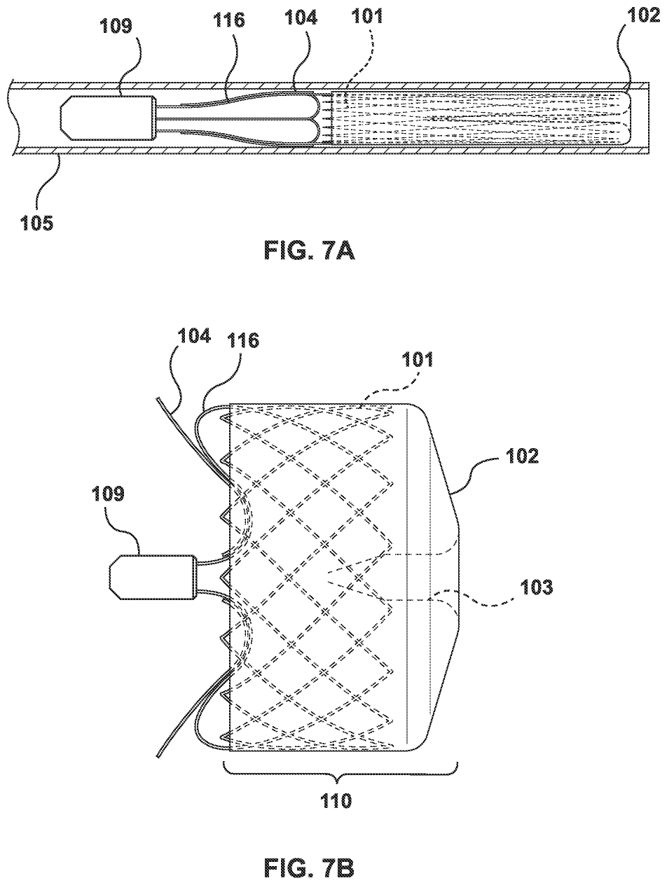

As shown in FIG. 7A in a constrained delivery state the barbs 104 may be retracted and flush with the spokes 116 and braided airway contact region 110, allowing the device to be advanced through or from a delivery sheath 105. When the lobar valve expands to in deployed state (FIG. 7B) the barbs 104 deploy to radially protrude from the airway contact region 110. The wires forming the barbs 104 may be connected to the spokes 116, for example, woven or bonded to the spokes. Alternatively, the wires forming the barbs 104 may be connected into the coupler 109, or a combination of connection to a coupler and connection to the spokes.

Alternatively, barbs 104 may be made from a laser cut hypotube. For example, a coupler, spokes and barbs may be made from a laser cut hypotube, wherein the spokes are connected to a braided structural frame forming an airway contact region.

Regardless of the retention mechanism embodied, a lobar valve 100 may be implanted and before removing the delivery tool and bronchoscope, a pull force test may be applied to the device to ensure it has been sufficiently anchored in place. With the delivery tool connected to a grasping mechanism of an implanted lobar valve, the pull force may be conducted by applying a gentle pull force on the delivery tool. A force gauge may indicate the amount of force applied to the lobar valve. If the valve becomes dislodged below a predetermined force, the retention mechanism of the stent may not suit the current implantation, a different sized device may be required, or the device may need to be repositioned.

### Example Embodiment 1 Braided Frame with Spokes

A first embodiment of a lobar valve as shown in FIGS. 2A, 2B, 5A, 5B, 6A, 6B, 7A and 7B has a braided structural frame 101 with an airway contact region 110 and radial spokes 116 connecting the airway contact region 110 to a coupler 109. A sealing membrane 102 is connected to the airway contact region 110 and extends past the distal end 115 of the airway contact region where it forms a luminal covering region 119 extending from diameter 111″ to diameter 121 and further forms a flow control valve 103. General features of these elements that are disclosed herein may apply to this embodiment.

The wires of the braided structural frame 101 have closed loop ends 113 on the distal side 115 and on the proximal side 114 the wires are gathered and shape-set to form the spokes 116. The terminals of the wires are held (e.g., crimped, welded) in the coupler 109.

Barbs 104 radially protrude from the proximal end of the airway contact region 110.

The spokes 116 may be angled proximally 114 as shown in FIG. 2A or alternatively may be perpendicular to the airway contact region 110 or angled distally as shown in FIG. 5A. Lobar valves with spokes that are angled distally position the coupler 109 at least partially in the lumen of the airway contact region 110 effectively reducing the device length 112″, which may be advantageous especially where less room in the airway is available. Inverted, or distally angled spokes may further facilitate retention.

Optionally spokes 116 may be “S” shaped spokes 155 as shown in FIG. 5B.

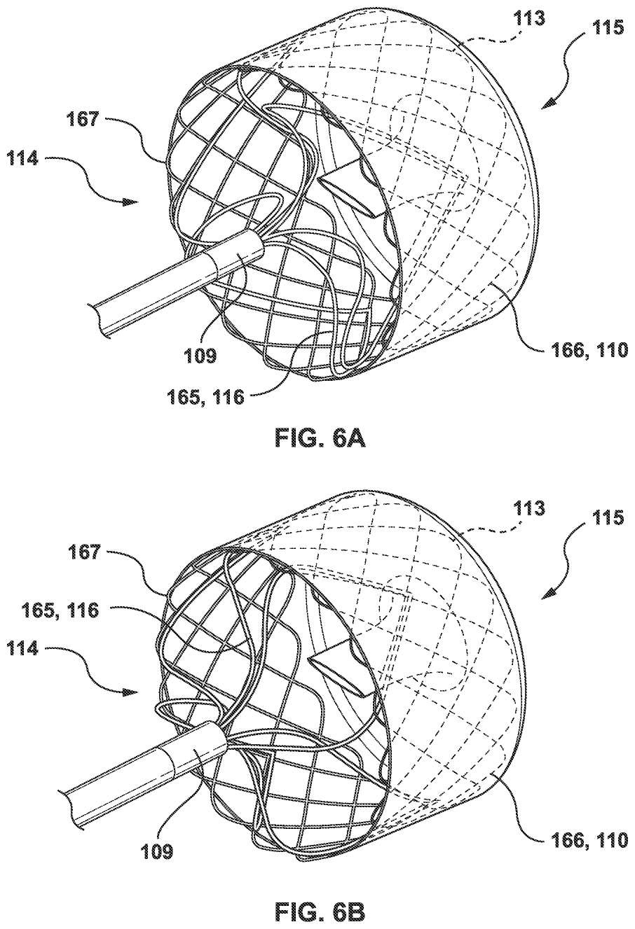

Alternatively, as shown in FIGS. 6A and 6B spokes 116 may be separate wires (e.g., Nitinol) 165 than the wire(s) forming the airway contact region 110, 166 of the structural frame. A tubular wire braided airway contact region 110, 166 may be made with a single wire with the terminals woven into the airway contact region 166 so that both distal 115 and proximal 114 ends have closed loop ends 113, 167. For example, a lobar valve may have separate wire spokes 165 including three wires (FIG. 6A) or four wires (FIG. 6B). The wires forming the spokes 165 may be looped through a part (e.g., proximal closed loop ends 167, or other part) of the braided airway contact region 166 with both terminals connected to the coupler 109.

Alternatively, spokes and optionally a coupler or radial barbs may be made from a laser cut hypotube (e.g., Nitinol).

The optional barbs 104 may be formed from a variety of options disclosed herein such as separate wires from the braided structural frame connected to the spokes or airway contact region, wires forming the braided structural frame cut and shape set to protrude forming the barbs, or portions of the braided structural frame shape set to protrude outward.

### Example Embodiment 2 Braided Frame with Tapered Proximal End

An alternative embodiment 180 of a lobar valve is shown in FIG. 8, wherein a structural frame 181 is made from a braided wire Nitinol tube that is shape set to form an airway contact region 182 having a first diameter 183, a valve housing region 185 having a narrower second diameter 186, and a luminal covering region 184 spanning from the first diameter 183 to the second diameter 186. The proximal end of the braided tube may be connected to a coupler 109 at the proximal end of the device 114. Enlarged holes 187 may be shape set into the braided tube 181 proximal to the valve housing region 185, which may facilitate flow of air or fluids or reduce the risk of clogging the braided frame. The luminal covering region 184 of the braided frame may have an “S” shaped profile as shown, which may facilitate deployment, retraction, and radial retention force. The membrane 188 may be connected to the airway contact region 182 of the braided frame, be open at the distal end 115 of the device 180, be connected to or at least span the luminal covering region 184, and form a flow control valve 189 that is contained in the valve housing region 185. Radially protruding barbs 190 optionally may be connected to the structural frame and may be a variety of barb embodiments or positions disclosed herein.

### Example Embodiment 3 Braided Frame Open on Both Ends

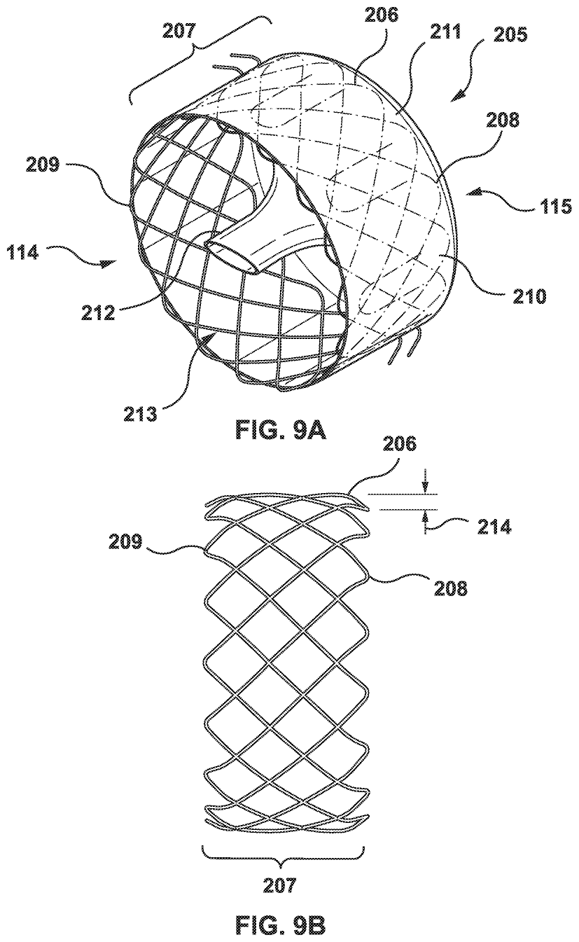

Another alternative embodiment of a lobar valve 205 is shown in FIG. 9A, wherein a braided structural frame 206 forms a tubular airway contact region 207 with an open distal end 115 and open proximal end 114. The distal end of the braid 115 and the proximal end of the braid 114 both may have closed loop ends 208 and 209. The braid 206 may be made from one wire with both terminals woven into the airway contact region 207. Optional radially protruding barbs 210 may be connected to the braided structural frame 206. For example, a pair of barbs 210 may be made from a Nitinol wire that is woven into the braid 206 with terminals shape set to radially protrude as shown. A membrane 211 may be connected (e.g., solvent bonded, dip coated, glued, sewn) to the airway contact region 207 of the braided structural frame 206, span a luminal covering region, and form a flow control valve 212 held in a lumen 213 defined by the airway contact region 207 of the structural frame 206.

Optionally, as shown in FIG. 9B the distal and proximal closed loop ends 208 and 209 of the braided structural frame 206 may be bent inward a distance 214 (e.g., 0.25 mm to 1 mm) as shown which may reduce a risk of irritating the tissue of the airway wall by reducing friction applied to the tissue by the closed loop ends during movement.

### Example Embodiment 4 Braided Frame Closed on Both Ends

Another alternative embodiment of a lobar valve shown in FIG. 10 has wire braided structural frame 231 having a tubular airway contact region 232 with a first diameter 233 adapted for placement in a lobar bronchus. At the proximal end 114 of the device 230 the wires of the braided structural frame 231 are shape set to span the luminal covering region 234 from the first diameter 233 to a narrower second diameter 235 where the wires are connected to (e.g., crimped into or welded to) a coupler 109. The wire braid in the luminal covering region 234 may have shape set holes 236 that may facilitate passage of air or other fluids. Similarly, at the distal side 115 of the device 230 the wires of the braided structural frame 231 span a luminal covering region 238 from the first diameter 233 to a narrower third diameter 237 where the wires may be crimped together in a distal crimp 239. The distal luminal covering region 238 optionally may also have shape set holes 240 to facilitate passage of air or other fluids. The proximal and distal luminal covering regions 234, 238 with shape set holes 242, 240 are an alternative form of spokes that connect the airway contact region to a hub (e.g. coupler 109 or crimp 239. Alternatively, spokes may be configured in other various embodiments of spokes disclosed herein which may be both on the proximal 114 and distal ends 115.

A sealing membrane 241 may be connected to the braided structural frame at least partially over the airway contact region 232 and a portion of the proximal luminal covering region 234 leaving an uncovered part 242 of the luminal covering region 234. A separate membrane flap 243 connected to the coupler 109 or structural frame temporarily covers the gap 242 and overlaps a portion of the membrane 241 when air pressure is higher on the proximal end 114 than the distal end 115. The flap 243 opens when pressure is higher on the distal end 115 than the proximal end 114. Thus, the flap 243 and membrane 241 act as a flow control valve.

Alternatively, the membrane 241 may partially cover the distal luminal covering region 238 and a flow control valve may be formed with a flap at the distal end also adapted to preferentially allow air flow from the distal to proximal ends (not shown).

Optionally, the membrane 241 at least on the exterior portion of the airway contact region 232 may have a molded micropattern 244 to increase retention in the airway.

Optionally, radially protruding barbs 245 may be connected to the braided structural frame 241. The barbs 245 may be one or more of the various embodiments of radially protruding barbs disclosed herein.

The braided structural frame forming both proximal 234 and distal 238 luminal covering regions may have increased strength or radial contact force with the airway wall in situ.

### Example Embodiment 5 Braided Frame Forming an Inner and Outer Tube

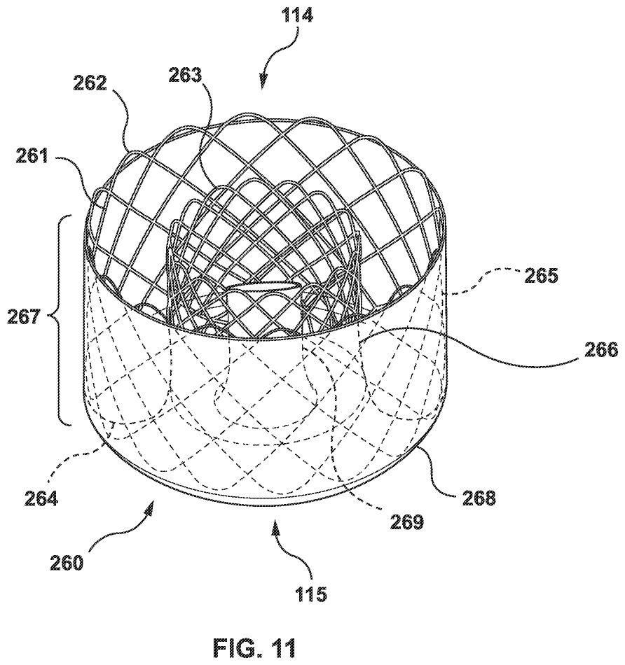

Another alternative embodiment of a lobar valve 260 is shown in FIG. 11 wherein a tubular braided structural frame 261 is a tube with a first end 262 and a second end 263 that is folded in on itself forming an outer tubular region 265 and an inner tubular region 266 spanned by a luminal covering region 264 on the distal end 115 of the device 260. Alternatively, a luminal covering region may be on a proximal end 114. The outer tubular region 265 forms an airway contact region 267. Optionally the tubular braided frame 261 may be made from one Nitinol wire braided into a tube that is shape set to form the outer and inner tubular regions 265, 266 and the wire terminals may be woven into the tube so they are not exposed. Optionally both the first end 262 and second end 263 may have closed loop ends. Optionally, at least some of the closed loop ends on the first end 262 may be bent outward to function as radially protruding retention barbs. Alternatively or optionally, radially protruding retention barbs may be made by cutting some of the closed loop ends on the outer first end 262, or made by connecting separate wires to the structural frame.

A sealing membrane 268 may be connected to the braided structural frame 261 on at least a portion of the airway contact region 267, where the membrane may optionally have a micropatterned surface on the exterior of the membrane to enhance retention in an airway. The membrane may also cover the luminal covering region 264 and form a flow control valve 269 (e.g., Heimlich or duckbill valve) in a lumen defined by the inner tube 266.

The embodiment shown in FIG. 11 may not have a separate component for a coupler but instead the second end 263 may connect to a delivery tool for delivery, deployment, or retraction of the device 260.

Optionally, a thread or suture may be woven into the proximal end of the frame. The thread may be used to contract the structural frame, for example the thread may be grasped with bronchoscopic forceps and pulled to contract the proximal end of the support frame to pull the device into a delivery sheath for initial loading or re-positioning. Optionally, the thread may be connected to a ball (e.g., threaded through a hold in the ball) that functions as a grasping element. The ball may be relatively small, e.g., between about 1 mm and 3 mm.

### Example Embodiment 6 Folding Braided Frame

An exemplary embodiment of a lobar valve 300 is shown in a cross-sectional illustration in FIG. 13, wherein the lobar valve has a structural frame that is a braided Nitinol tubular structure having two folds in its expanded unconstrained configuration. The two folds may have improved ability to remain anchored in place when implanted for example by applying improved outward radial force to the bronchus wall, greater friction between the membrane (optionally with a micropattern) and the bronchus wall, or improved force that maintains the frame in an expanded configuration to resist deformation that may allow the lobar valve from being displaced from its intended implant location distal to a cartilage ring and proximal to a carina.

FIG. 13 shows the folding lobar valve 300 in an unconstrained expanded configuration. The lobar valve 300 is generally radially symmetric about a central axis 306. The lobar valve 300 has a proximal end 301 and a distal end 302 and the lobar valve is intended to be implanted so that exhaled air flows out of the lung in a direction from the distal end toward the proximal end 301 as illustrated with airflow arrows 120. The lobar valve 300 has a braided Nitinol structural frame 303, which may have features of structural frames disclosed herein, a coupler 304, which may have features of coupler 109 disclosed herein (e.g., FIG. 4), a membrane 305, which may have features of membrane 102 disclosed herein, and a valve 307, which may have features of valves 103 disclosed herein.

The braided frame 303 is generally tubular or cylindrical in shape and has a wall contact section 310, a first fold 311 on the proximal side of the wall-contact section, a middle support section 312 that fits inside the wall contact section, a second fold 313, and an optional inner support section 314 that fits inside the middle support section. The wall-contact section 310, first fold 311, middle support section 312, second fold 313 and, optional inner support section 314 may be fabricated by braiding at least one Nitinol wire that is superelastic at body temperature. The folds may be fabricated by shape-setting the Nitinol wire or Nitinol braid.

Optionally the folds 311 or 313 may have a braid angle that is different than the braid angle in the wall contact section 310, middle support section 312 or inner support section 314.

The wall-contact section 310 has a length 316 in a range of 8 mm to 18 mm. The middle support section 312 may have a length 317 that is shorter than the wall-contact section length 316. For example, in the expanded configuration shown in FIG. 13A the middle support section 312 has a length 317 that is less than the length 316 of the wall-contact section 310 so that the middle support section resides both radially and longitudinally within the wall-contact section. The inner support section 314 may have a length 318 that is shorter than the middle support section length 317. For example, in the expanded configuration shown in FIG. 13A the inner support section 314 has a length 318 that is less than the length 317 of the middle support section 312 so that the inner support section resides both radially and longitudinally within the middle support section. Optionally, the length 317 of the middle support section may be half or less of the length 316 of the wall contact section, which may apply added radial or retention force predominantly to the proximal half or less of the wall-contact section where it may be most beneficial for retention distal to a cartilage ring of the bronchus.

The distal end of the wall-contact section may comprise closed end loops 319 formed by the Nitinol wire. Optionally, the closed end loops 319 may have features of closed end loops disclosed elsewhere such as 125 shown in FIG. 3B or 135 and 136 shown in FIG. 3A. Optionally, the closed end loops 319 may be bent inward toward the central axis 306 of the lobar valve, which may favorably reduce wear on the membrane where it contacts the end loops or may improve ability to collapse the lobar valve into a delivery sheath as shown in FIG. 13C. Distal ends that are bent inwards may also provide a less traumatic end that will be less susceptible to creating an inflammatory response during ventilation and subsequently less granulation tissue over time.

Embodiments having the inner support section 314, may have spokes 315 connecting the proximal end of the inner support section 314 to the coupler 304. The spokes may be fabricated from the same wires that are braided to form the inner support section and rest of the braided frame. For example, the wires continuing from the braided the inner support section may be bundled into groups (e.g., groups of 3, of 4, of 5, of 6) to form the spokes. There may be a total of 3 to 15 spokes for example. The bundled groups of wires have adequate strength and stiffness so force applied at the coupler by a delivery tool can push the device out of a delivery sheath, pull the device back into a sheath, or manipulate the placement of the device. Also, bundling the wires at this location provides large spaces 321 between the spokes 315 at the proximal end of the device where the membrane is not located, which can allow fluid such a mucus to escape from the space in the device. The spokes 315 may have a length when straightened (see FIG. 13C) in a range of 5 to 15 mm. For example, the spokes may have a length that is in a range that is equal to a radius of the targeted bronchus to about 3 mm greater than the radius of the targeted bronchus. The spokes may have features disclosed herein for other spokes 116 such as an S-curve.

A similar embodiment 324 of a lobar valve but without an inner support section is shown in FIG. 13B wherein the same callout numbers are used as in FIG. 13A for all other components. The spokes 315 connect the coupler 304 to the second fold 313 instead of an inner support section. In one example of this embodiment the second fold 313 is formed from braided wires that are bundled proximal to the second fold 313. Alternatively, the second fold 313 may be formed from bundled wires.

Optionally, barbs may be connected to the braided Nitinol support structure 303 or fabricated from some of the wires forming the support structure 303. The retention elements may have features of barbs 104 disclosed herein.

The coupler 304 may be connected to the spokes 315, for example by crimping the bounded wires forming the spokes into the coupler. The coupler is adapted to be releasably connected to a delivery tool, for example via mating threads or mating geometry. The coupler 304 may have other features disclosed herein for couplers 109.

The membrane 305 is connected to the outward face of the wall contact section 310 of the braided Nitinol frame 303 and extends at the distal end 302 of the device inward toward the central axis 306 to block the lumen in the airway and hold a flow control valve 307 in the airway lumen. Airflow through the airway lumen is directed by the membrane 305 through the flow control valve 307. The valve and membrane may be the same material and component. The configuration shown in FIG. 13A with the membrane obstructing the airway at the distal end 302 of the flow control device 300 and the proximal end 301 remaining open may allow a pressure differential between the distal and proximal sides of the device 300 that is created when the patient inhales create a billowing effect on the membrane wherein the membrane is pushed against the airway wall to produce a better seal during inhalation. The membrane 305 may be bonded to the wall-contact section 310 of the structural frame 303, for example via over molding or adhesion with a bonding substrate. Alternatively, the membrane may be bonded only in certain spots of the wall-contact section 310 that comprise less than 100% of the wall-contact section, which may reduce the force loading to allow the structural frame 303 to transition from its contracted delivery state to its expanded state more easily. For example, the membrane may be bonded to one or more portions of the wall-contact section in longitudinal strips, circumferential bands, or spots and the bonding may comprise a portion of the wall-contact section in a range of 25% to 100%, (e.g., 25% to 80%, 30% to 75%, 30% to 60%). Optionally, the membrane is not bonded to the distal end (e.g. distal end loops or distal few mm) of the structural frame 303, which may further improve ability to transition between the contracted and expanded states. One way of preventing bonding of the membrane at the distal end of the frame is to shape set the distal end tips inwards so that the membrane is physically away from the frame during a bonding process. The membrane 305 may have features disclosed herein for other membranes 102 such as a hydrophilic micropattern on the outer surface 322 (e.g., at least of the portion of membrane covering the wall-contact section 310 of the frame 303), a hydrophobic micropattern on the inner surface 323.

The valve 307 (FIG. 13A) is positioned at the distal side 302 of the device 300 and may have features of valves 103 disclosed herein. For example, the valve 307 may be made from the membrane material and be formed as part of the membrane during a molding process. The valve 307 may be a duckbill or Heimlich valve that allows air to flow, at least predominantly, in the exhale direction.

FIG. 13C shows the lobar valve 300 (aka flow control device 300) in its contracted state constrained in a delivery sheath 105. The braided Nitinol structural frame 303 is essentially unfolded so the coupler 304, spokes 315, optional inner support section 314, second fold 313, middle support section 312, first fold 311, and wall-contact section 310 are adjacent to one another. The coupler 304 is shown connected to a distal end of a delivery tool 108. The spokes 315 in the contracted state have a length 320, which is longer than the length 318 of the inner support section and the length 317 of the middle support section, and less than the length 316 of the wall support section 310.

A method of implanting the flow control device 300 may comprise advancing a delivery sheath 105 through the patient's airway to a target lobar bronchus; advancing the flow control device 300 partly from the delivery sheath to deploy the wall contact section 310 and optionally the first fold 311; placing the wall contact section in the target lobar bronchus and allowing the wall contact section to contact a wall of the lobar bronchus (adjustments in position may be made by advancing or retracting the delivery sheath 105 while keeping the delivery tool 108 and portion of the lobar valve 300 stationary with respect to the delivery sheath); then advancing the flow control device further from the delivery sheath to deploy the first fold, the middle support section, and the second fold. If the flow control device 300 further comprises an inner support section 314, the inner support section may also be advanced from the sheath following the step of allowing the wall contact section to contact the wall of the lobar bronchus. If the targeted lobar bronchus has a cartilage ring at its proximal end, the step of placing the wall contact section in the target lobar bronchus may comprise placing the wall contact section distal to the cartilage ring and optionally placing the first fold 311 distal and adjacent to the cartilage ring. The flow control device may be advanced from the sheath by pushing the flow control device with a delivery tool or holding the delivery tool stationary with respect to the patient's lung and retracting the delivery sheath. The delivery tool may be coupled to the flow control device, for example with screw threads, and may be decoupled from the flow control device, for example by rotating the delivery tool to unscrew it from the coupler, while the flow control device is held in place by the targeted airway.

### Example Embodiment 7 Folding Braided Frame with Locking Ring

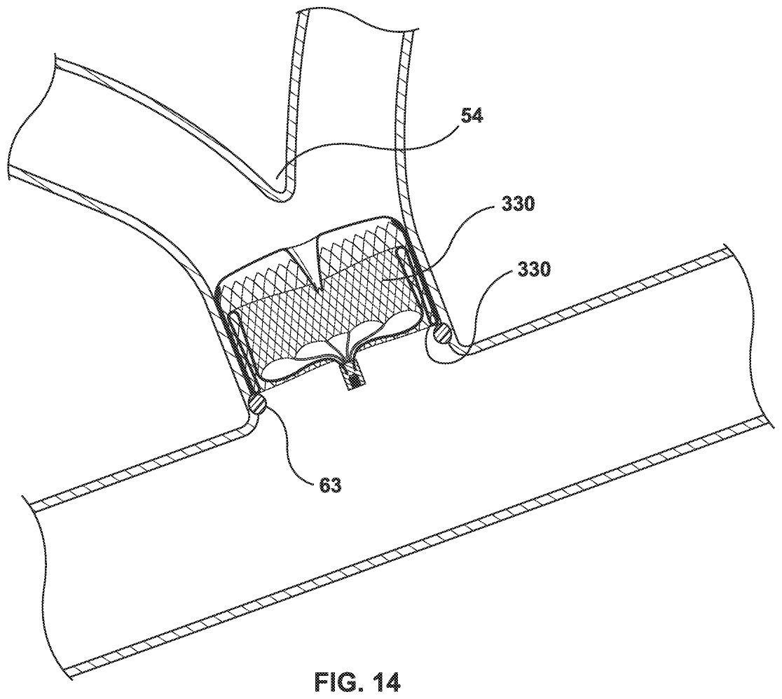

As shown in FIG. 14, a variation on the folding frame embodiment 300 comprises a locking ring 330 intended to be placed distal and adjacent to the cartilage ring creating a mechanical feature that will further anchor the frame in place resisting proximal migration. This locking ring is essentially the first fold 311 of the embodiment 300, however the first fold may have a particularly tight bend radius (e.g., a radius that is less than or equal to the height of the cartilage ring, a radius in a range of 0.75 mm+/−0.5 mm) so it wedges behind the cartilage ring and doesn't slip off.

Optionally, the braid angle at the first fold 311 or locking ring 330 may be larger than the braid angle in the flat sections (e.g., wall-contact section 310, middle support section 312, or inner support section 314) of the frame 303. The larger braid angles at the folds may provide the following: greater radial forces that may provide a better retention feature; and lower bend radii at the folds that may make it easier to collapse the device and load it into a delivery sheath.

Optionally, the first fold 311 or second fold 312 may be made during a shape set process by bending the wires around mandrels.

Another characteristic of the device shown in FIG. 14 is that other parts of the device are sized to be apart from the first fold/locking ring 330 so as to not interfere with the function of the locking ring 330. For example, the inner support section 314 may be shorter than the middle support section 312 as shown so that the inner support section or spokes do not contact the cartilage ring 63.

Delivery Tool

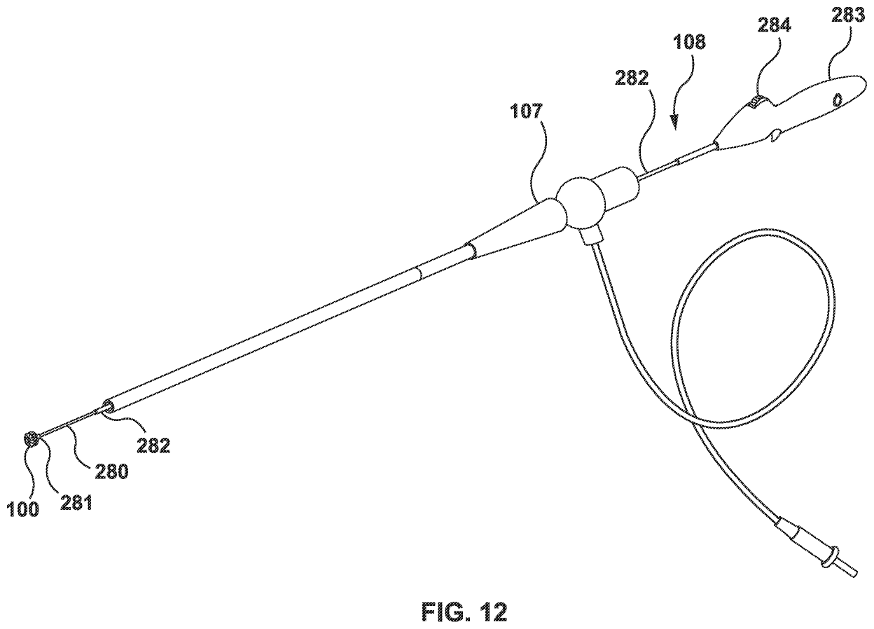

As shown in FIG. 12 a delivery tool 108 for delivering a lobar valve (e.g., 100) through a working channel of a bronchoscope 107 may have a delivery shaft 280, which may be a flexible, elongate, tubular or rod structure, with a coupling element 281 at its distal end that is shaped to couple with the coupler (e.g., 109) of the lobar valve, a delivery sheath 282, and a handle 283 at its proximal region. For example, the coupling element 281 may be a male threaded rod adapted to mate with a female threaded opening 145 of a lobar valve coupler 109 (FIG. 4). A delivery shaft may be flexible to bend and navigate through a bent bronchoscope in a tortuous airway yet be longitudinally and circumferentially non-compliant to resist stretching, compression or kinking so it transmits motion from the proximal end (e.g., handle) to the coupling element 281 and to the lobar valve 100. A delivery shaft may be made from a polymer and have an embedded laser cut tube or tight wire coil. Optionally, the delivery shaft 108 may have a visual depth marker on its proximal region that lines up with the proximal opening of the delivery sheath 105 when the lobar valve is just inside the sheath.

An alternative embodiment of a delivery shaft, may have a central lumen, which may be used for delivery over a guidewire or to pass over or deliver other instruments such as an endoscope. Optionally a delivery shaft may have a mandrel extending distally, which may be used to hold a valve to the delivery shaft, to add coupling force, to target a coupler of a lobar valve when retrieving it or to adjust its position.

Optionally, the delivery tool may have a delivery sheath 282 used in conjunction with the delivery shaft 280. The sheath may constrain the valve in a delivery state during delivery through a working channel as shown in FIG. 2B. A distal section (e.g., about 10 cm of the distal end) of the delivery sheath may be relatively more flexible allowing it to bend and traverse a bronchoscope that is bent at its distal end to navigate a tortuous airway. The delivery sheath may be non-compliant over its full length to resist compression or stretching. The delivery sheath may be circumferentially non-compliant at least at its distal end so it can contain and constrain a lobar valve in its contracted delivery state. A laser cut steel tube may be embedded in a polymer such as Pebax at its distal section to provide hoop strength and circumferential non-compliance. The delivery sheath may be made of a polymer such as Pebax or polyimide with an embedded wire braid or wire coil to resist compression, stretching or kinking. The delivery sheath 282 may have an outer diameter sized to slidably pass through the bronchoscope working channel 106 (e.g., to fit a 2.8 mm the sheath may have an outer diameter in a range of 2.0 mm to 2.7 mm). The sheath may have an inner diameter in a range of 1.5 mm to 2.5 mm. Optionally, the delivery sheath 282 may have a visual depth marker on its proximal region that lines up with the proximal opening of the bronchoscope 107 when the distal end of the delivery sheath is aligned with the distal end of the bronchoscope. The marker 357 may be 5 to 15 cm from the proximal end of the sheath so the distal end of the sheath can be further advanced from the bronchoscope. The visual depth marker may allow a user to quickly advance the delivery sheath through the bronchoscope providing visual indicator of when the distal end of the sheath is approaching the distal opening of the bronchoscope.

Optionally, the delivery tool may have a handle 283 at a proximal region that has an actuator (e.g., thumb lever) that controls a sliding translational movement of the shaft 280 with respect to the sheath 282 facilitating one-handed control for advancing a valve out of a sheath or retracting it into the sheath. For example, a sheath 282 may be connected to the handle body and a shaft 280 may be slidably engaged in the sheath and connected to a gear that is movable (e.g., rotation or translation) within the handle and moved by a mating gear connected to an actuator such as a thumb lever, slider, or rotary dial. The handle may have one or more actuators that move the delivery shaft and control the position of the lobar valve from a fully contained position as shown in FIG. 2B to a partially deployed position with the couplers connected to a fully deployed and released position as shown in FIG. 2C. A first actuator 284 may be used to pull the device 100 into the delivery sheath (FIG. 2B), optionally with help of a loading tool. The first actuator may be used to advance and retract the sheath 282 relative to the shaft 280 to partially deploy the lobar valve 100. This step may be used to assess position and fit within a target airway while visualizing deployment through a lens of the bronchoscope 107. The first actuator 284 may stop at the position of stage 1 before fully releasing the device. A second actuator 285 such as a trigger may be used to fully release the lobar valve 100, for example by rotating the shaft 280 to unscrew the delivery tool coupler 281 from the device coupler 109. The first and second actuators may be ergonomically oriented on the handle 183 to be used with one hand for example the first actuator may be oriented for use with a thumb and the second actuator may be oriented for use with an index finger of the same hand.

Kit

Optionally a lobar valve may be provided preloaded in a delivery sheath, optionally disposable, in its constrained delivery state and coupled with a delivery shaft as shown in FIG. 2B. Alternatively, a lobar valve may be provided coupled to a delivery tool with the lobar valve advanced out of a delivery sheath in its unconstrained state for example as shown in FIG. 2A. The assembly may be provided contained in a sterilized package with instructions for use. A lobar valve provided partial deployed may facilitate visual inspection and avoid material deformation caused by prolonged constraint. Optionally, the unconstrained lobar valve may be held in a loading tool, for example having a funnel, that facilitates contraction of the device into a contracted delivery state in a delivery sheath.

Delivery

A method of use may involve the following delivery steps:

From a CT scan, measurements are taken to confirm intended valve placement location, target airway diameter and length;

An appropriately sized lobar valve is chosen to match the measured airway size.

The lobar valve is visually inspected prior to loading into a delivery sheath;

A bronchoscope is advanced through the patient's endotracheal tube to the targeted lobar airway;

The lobar valve in the delivery sheath is advanced distally through a working channel of the bronchoscope;

The distal end of the delivery system is advanced distally out of the working channel to a desired valve position in the target airway;

Optionally, aligning a radial visual marker on the delivery sheath with an orientation of an oval bronchus.

While holding the bronchoscope in position relative to the airway the delivery sheath is retracted proximally relative to the shaft and lobar valve to deploy the lobar valve to its expanded but coupled position;

The position, fit, alignment, and seal may be visually inspected through the lens of the bronchoscope. The delivery system may be pulled gently to confirm mechanical anchoring or engagement of the valve against the airway wall;

If position, fit, alignment, seal and anchoring are not satisfactory optionally push or pull the delivery system to adjust;

If position, fit, alignment, seal and anchoring are still not satisfactory retract the lobar valve at least partially back into the delivery sheath;

The delivery sheath and lobar valve may be repositioned and redeployed;

If the position, fit, alignment, seal and anchoring are satisfactory the lobar valve may be disengaged from the coupler of the delivery system;

The delivery system may be removed from the patient;

The lobar valve may be visually inspected through the lens of the bronchoscope;

The bronchoscope may be removed from the patient.

While at least one exemplary embodiment of the present invention(s) is disclosed herein, it should be understood that modifications, substitutions and alternatives may be apparent to one of ordinary skill in the art and can be made without departing from the scope of this disclosure. This disclosure is intended to cover any adaptations or variations of the exemplary embodiment(s). In addition, in this disclosure, the terms “comprise” or “comprising” do not exclude other elements or steps, the terms “a” or “one” do not exclude a plural number, and the term “or” means either or both. Furthermore, characteristics or steps which have been described may also be used in combination with other characteristics or steps and in any order unless the disclosure or context suggests otherwise. This disclosure hereby incorporates by reference the complete disclosure of any patent or application from which it claims benefit or priority.

1. A flow control device for a bronchial passageway comprising:
a flow control valve;
a braided wire structural frame, wherein the structural frame is expandable from a collapsed configuration to an expanded configuration, and in the collapsed configuration the frame is an extended tube and in the collapsed configuration the frame includes a wall contact section, a middle support section within the wall contact section, and a fold between and connecting the wall contact section and the middle support section; and
a sealing membrane mounted to at least a distal portion of the structural frame, wherein the sealing membrane forms an enclosed wall defining at least a portion of an airflow passage through the flow control device, and the flow control valve is included in the airflow passage and extending inward from the enclosed wall and at least partially within the wall contact section.

2. The flow control device of claim 1 wherein the flow control valve is integrated in the sealing membrane.

3. The flow control device of claim 1, further comprising a coupler and spokes extending radially outward from the coupler to a proximal end of the braided wire structural frame.

4. The flow control device of claim 1, wherein the wall contact section is longer than the middle support section.

5. The flow control device of claim 1, wherein the braded wire structural frame includes an inner support section connected to the middle support section by a second fold.

6. The flow control device of claim 5, wherein the middle support section is longer than the inner support section.

7. The flow control device of claim 5, wherein the inner support section is directly connected to spokes extending radially inward of the inner support section to a coupler.

8. The flow control device of claim 1, wherein a width of the braided wire structural frame, in the expanded configuration is in a range of 7 mm to 18 mm.

9.-42. (canceled)

43. An assembly of an air flow control devices and an insertion tool for a bronchial passageway comprising:
air flow control device, wherein each of the air flow control devices includes:
a flow control valve;
a braided wire structural frame, wherein the structural frame is expandable from a collapsed configuration to an expanded configuration and the braided wire structural frame in the collapsed configuration is an elongated tube and in the expanded configuration includes a wall contact section, a middle support section, residing radially within the wall contact section, and a first fold between the wall contact section and the middle support section;
a sealing membrane mounted to at least a distal portion of the structural frame, wherein the sealing membrane forms an enclosed wall defining at least a portion of an airflow passage through the flow control device, and the flow control valve is included in the airflow passage, and
a first coupler at a proximal end of the airflow control device;
a delivery sheath configured to be positioned in a bronchial passageway, wherein the delivery sheath includes a distal end, wherein the air flow control device, while in the collapsed configuration, is within the delivery sheath;
a delivery shaft within the delivery sheath and extends through the delivery sheath towards the distal end; and
a second coupler at the distal end of the delivery shaft, wherein the second coupler is configured to securely engage the first coupler,
wherein the delivery shaft is configured to advance through the delivery sheath to push the air flow control device from the distal end of the delivery sheath and into the bronchial passageway,
wherein the air flow control device is configured to expand from the collapsed configuration into the expanded configuration after the air flow control device is pushed out of the delivery sheath, and
wherein the air flow control device is configured to automatically release from the second coupler when an actuator on a handle of the assembly is actuated.

44. The assembly of claim 43, further comprising a visual marker on a distal region of the delivery sheath, wherein the visual marker indicates an angular position of a semi-minor or semi-major axis of the braided wire structural frame.

45. An implantable airflow control device for a lobar bronchus comprising a distal end and a proximal end, a braided Nitinol frame, and a membrane affixed to a distal end of the frame, and wherein the airflow control device expands from a collapsed state to an expanded state, and the frame in the collapsed configuration is an elongated tube and in the expanded configuration includes a wall contact section, a middle support section residing radially within the wall contact section, and a first fold between the wall contact section and the middle support section.

46. The device of claim 45, wherein the braided frame, comprises a wall contact section, a first fold, a middle support section residing radially within the wall contact section, and a second fold.

47. The device of claim 46, wherein the middle support section resides radially within the wall contact section when the device is in its expanded state and adjacent to the wall-contact section when the device is in its collapsed state.

48. The device of claim 46, wherein the middle support section is shorter than the wall contact section.

49. (canceled)

50. The device of claim 45, further comprising an inner support section resides radially within the middle support section when the device is in its expanded state and adjacent to the middle support section when the device is in its collapsed state.

51. The device of claim 45, wherein the inner support section is shorter than the middle support section.

52.-53. (canceled)

54. The device of claim 46, wherein the wall-contact section has a length in a range of 8 mm to 18 mm when the device is in its expanded state.

55. The device of claim 46, wherein the device has a diameter in a range of 7 mm to 12 mm or in a range of 5 mm to 15 mm or in a range of 11 mm to 14 mm or in a range of 10 mm to 18 mm when the device is in its expanded state.

56. The device of claim 46, wherein the device has a diameter in a range of 2 to 2.6 mm in its collapsed state.

57. The device of claim 46, wherein the device has a length to diameter ratio in a range of 0.28:1 to 0.54 to 1 in its expanded state.

58-153. (canceled)
